SEQUENCE LISTING (2 sequences)

SEQ ID NO: 1 - protein, 24 aa, Artificial Sequence
  Synthetic
  MISC_FEATURE: X-Ala-U-Val-Leu-Lys-O-U-Ile-Lys-Val-Ala-Lys-Lys -Tyr-Ala-Lys-Gly-V al-A*-Leu-A*-Ala-Gly-Ala-Asn-Ile-O-Gly-Gly-Lys; X is hydroxy propionyl (Hop), U is a,b-didehydroalanine (Dha), O is a,b-dehydrobutyrine (Dhb), A* is aminobutyrine (Abu)
AVLKIKVAKKYAKGVLAGANIGGK

SEQ ID NO: 2 - protein, 24 aa, Artificial Sequence
  Synthetic
  MISC_FEATURE: X-ala-U-Ile-Val-Lys-O-O-Ile-Lys-Ala-Ala-Lys-Lys -Leu-Ala-Arg-Gly-P he-A*-Leu-A*-Ala-Gly-Ala-His-Phe-O-Gly-Lys-Lys; X is hydroxy propionyl (Hop), U is a,b-didehydroalanine (Dha), O is a,b-didehydrobutyrine (Dhb), A* is aminobutyrine (Abu)
AIVKIKAAKKLARGFLAGAHFGKK

(19) United States
(12) Patent Application Publication
(10) Pub. No.: US 2007/0259406 A1
(43) Pub. Date: Nov. 8, 2007

(54) Antimicrobial Compounds

(76) Inventors: Jan Verhoef, Zeist (NL); Danica Milatovic, Zoetermeer (NL); Miquel Ekkelenkamp, Utrecht (NL)

Correspondence Address: Medlen & Carroll, 101 Howard Street, Suite 350, San Francisco, CA, 94105, US

(21) Appl. No.: 10/571,572

(22) PCT Filed: Sep. 13, 2004
(86) PCT No.: PCT/EP04/10317

§ 371 Date: Jan. 22, 2007

(30) Foreign Application Priority Data
Sep. 11, 2003 (EP) PCT EP03 10188

(51) Int. Cl. A61K 38/16; A61P 31/04; C07K 14/195; C12P 21/04

(52) U.S. Cl. 435/71.3; 514/12; 530/324

(57) ABSTRACT

The present invention relates to new antimicrobial compounds, in particular proteins, having the amino acid sequence XAlaUValLeuLysOUIleLysValAlaLysLysTyrAlaLysGlyValA*LeuA*AlaGlyAlaAsnIleOGlyGlyLys wherein X is hydroxy propionyl (Hop), U is α,β-didehydroalanine (Dha), O is α,β-didehydrobutyrine (Dhb), A* is aminobutyrine (Abu), wherein lanthionine ring structures are formed between Ala12 and Ala16, between Abu20 and Ala23 and between Abu22 and Ala25, and in which at least one amino acid has been replaced by another amino acid.

The present invention relates to new antimicrobial compounds, in particular proteins and peptides, that are derived from a novel protein that was isolated from Staphylococcus epidermidis. The invention also relates to fragments of these proteins and peptides and to modified versions of the proteins and fragments thereof that display a similar and preferably at least the same antimicrobial activity as the isolated protein. The invention further provides the compounds for use in antimicrobial therapy and the use of the compounds for the preparation of a pharmaceutical preparation for the treatment of microbial infection.

Antibiotics are commonly used in the treatment and/or prevention of infectious diseases caused by various microorganisms. However, the spread of bacterial resistance leads to a growing demand for novel antibiotics.

The object of the present invention is therefore to provide new antimicrobial agents.

In the research that led to the present invention an antimicrobial protein having a molecular weight of about 3100 kDalton was obtained by the following method:

a) growing Staphylococcus epidermidis strain 15x154 (CBS accession no. 113428) in growth medium;

b) removing the cells for obtaining the supernatant;

c) passing the supernatant over a cation exchange liquid chromatography column;

d) eluting fractions and determining their antimicrobial activity;

e) pooling the fractions showing antimicrobial activity and passing them over a hydrophobic interaction column;

f) eluting fractions and determining their antimicrobial activity;

g) pooling the fractions showing antimicrobial activity and passing them over a reverse phase liquid chromatography column;

h) eluting fractions and determining their antimicrobial activity; and

i) pooling the active fractions and concentrating the protein contained therein.

The columns are as used in Example 1. The protein is isolated from Staphylococcus epidermidis strain 15x154 (deposited at Centraal Bureau voor Schimmelcultures (CBS) on 9 Sep. 2003 under the accession number CBS 113428), which is a clinical isolate from a wound infection.

The activity of the fractions is tested against Staphylococcus epidermidis ATCC strain 49134. The protein of the invention is furthermore active in inhibiting the growth of the following microorganisms: coagulase negative staphylococci, Staphylococcus aureus, Enterococcus spp., Acinetobacter, Escherichia coli.

It was found that the protein thus isolated is a so-called lantibiotic. Lantibiotics are antibiotic peptides distinguished by the presence of the rare thioether amino acids lanthionine and/or methyllanthionine. They are produced by Gram-positive bacteria as gene-encoded precursor peptides and undergo post-translational modification to generate the mature peptide. Based on their structural and functional features lantibiotics are currently divided into two major groups: the flexible amphiphilic type-A and the rather rigid and globular type-B. Type-A lantibiotics act primarily by pore formation in the bacterial membrane by a mechanism involving the interaction with specific docking molecules such as the membrane precursor lipid II.

The primary sequence and solution structure of the thus obtained novel lantibiotic, which was named epilancin 15X, from Staphylococcus epidermidis was determined by the combined use of high resolution NMR spectroscopy and mass spectrometry. The molecule contains ten modified amino acids, three lanthionine ring structures, and a hydroxy-propionyl N-terminal moiety. The primary sequence shows 68% sequence homology with the known lantibiotic epilancin K7. The molecular structure was found to be closely similar to that of K7.

Based on this finding a new class of antimicrobial compounds is defined which comprises antimicrobial compounds having the amino acid sequence XAlaUValLeuLysOUIleLysValAlaLysLysTyrAlaLysGlyValA*LeuA*AlaGly AlaAsnIleOGlyGlyLys

wherein X is hydroxy propionyl (Hop), U is ? ??-didehydroalanine (Dha), O is ? ??-didehydrobutyrine (Dhb), A* is aminobutyrine (Abu), wherein lanthionine ring structures are formed between Ala12 and Ala16, between Abu20 and Ala23 and between Abu22 and Ala25, and

in which at least one amino acid has been replaced by another amino acid.

In a particular embodiment of the invention an amino acid in position 4, 5, 8, 11, 15, 17, 19, 26, 27 or 30 is replaced by another amino acid. This leads to a series of compounds wherein the amino acids in any one of the combination of positions as given in Table 4 are replaced by other amino acids.

In a further embodiment the antimicrobial compounds of the invention comprise at least one of the following amino acid substitutions Val4Ile, Leu5Val, Dha8Dhb, Val11Ala, Tyr15Leu, Lys17Arg, Val19Phe, Asn26His, Ile27Phe, Gly30Lys, which leads to a series of compounds which comprise any one of the combination of amino acid substitutions as listed in Table 5.

A specific embodiment of the invention relates to the antimicrobial compound which is named herein 15X and which has the amino acid sequence XalaUIleValLysOOIleLysAlaAlaLysLysLeuAlaArgGlyPheA*LeuA*AlaGly AlaHisPheOGlyLysLys, wherein lanthionine ring structures are formed between Ala12 and Ala16, between Abu20 and Ala 23 and between Abu22 and Ala25.

The invention further relates to antimicrobial compounds having a sequence homology of at least 71% with the sequence of 15X as given above. Preferably the sequence homology is at least 74% or 77%, preferably at least 81% or 84%, more preferably at least 87% or 90%, even more preferably at least 94% or 97% with the sequence of 15X. Sequence homology as used herein is intended to mean the percentage of identical amino acids over the complete sequence of 15X.

The novel protein shows a broad spectrum antimicrobial activity as demonstrated by means of a standard micro-dilution panel, for example against Staphylococcus aureus, Staphylococcus epidermidis, methycillin resistant Staphylococcus aureus (MRSA), Enterococci and haemolytic Streptococci.

In some cases the potential for use of proteins in drugs may be limited for several reasons. Proteins may for example be to large or too hydrophilic to pass membranes like the cell-membrane and the blood-brain barrier, and may be rapidly excreted from the body by the kidneys and the liver, resulting in a low bioavailability. Furthermore, they may suffer from a poor biostability and chemical stability since they may be quickly degraded by proteases, e.g. in the gastro-intestinal tract. Also, proteins might assume various conformations. The bioactive conformation usually is only one of these possibilities, which sometimes might lead to a poor selectivity and affinity for the target receptor. Finally, the potency of the peptides may not be sufficient for therapeutical purposes.

As a result of the above described drawbacks, proteins are sometimes mainly used as sources for designing other drugs, and not as actual drugs themselves. In such case it is desirable to develop compounds in which these drawbacks have been reduced.

The invention therefore further relates to fragments and modified versions of the protein as claimed having at least essentially the same antimicrobial activity as the native protein. “At least essentially the same” in this respect means that the antimicrobial activity of the fragment or modified version should not be substantially lower than the activity of the native protein in isolated form.

Modifications can be amino acid changes that do not affect the antimicrobial activity but are for example intended to improve other properties of the protein such as half-life, solubility, clearance etc. and may comprise traditional amino acid substitutions or the use of peptidomimetic building blocks to replace part of the protein.

Various definitions for peptidomimetics have been formulated in literature. Among others, peptidomimetics have been described as “chemical structures designed to convert the information contained in peptides into small non-peptide structures”, “molecules that mimic the biological activity of peptides but no longer contain any peptide bonds”, “structures which serve as appropriate substitutes for peptides in interactions with receptors and enzymes” and as “chemical Trojan horses”.

In general, peptidomimetics can be classified into two categories. The first consists of compounds with non-peptide-like structures, often scaffolds onto which pharmacophoric groups have been attached. Thus, they are low molecular-weight compounds and bear no structural resemblance to the native peptides, resulting in an increased stability towards proteolytic enzymes.

The second main class of peptidomimetics consists of compounds of a modular construction comparable to that of peptides, i.e. oligomeric peptidomimetics. These compounds can be obtained by modification of either the peptide side chains or the peptide backbone. Peptidomimetics of the latter category can be considered to be derived of peptides by replacement of the amide bond with other moieties. As a result, the compounds are expected to be less sensitive to degradation by proteases. Modification of the amide bond also influences other characteristics such as lipophilicity, hydrogen bonding capacity and conformational flexibility, which in favourable cases may result in an overall improved pharmacological and/or pharmaceutical profile of the compound.

Oligomeric peptidomimetics can in principle be prepared starting from monomeric building blocks in repeating cycles of reaction steps. Therefore, these compounds may be suitable for automated synthesis analogous to the well-established preparation of peptides in peptide synthesizers. Another application of the monomeric building blocks lies in the preparation of peptide/peptidomimetic hybrids, combining natural amino acids and peptidomimetic building blocks to give products in which only some of the amide bonds have been replaced. This may result in compounds which differ sufficiently from the native peptide to obtain an increased biostability, but still possess enough resemblance to the original structure to retain the biological activity.

Suitable peptidomimetic building blocks for use in the invention are amide bond surrogates, such as the oligo-β-peptides (Juaristi, E. Enantioselective Synthesis of b-Amino Acids; Wiley-VCH: New York, 1996), vinylogous peptides (Hagihari, M. et al., J. Am. Chem. Soc. 1992, 114, 10672-10674), peptoids (Simon, R. J. et al., Proc. Natl. Acad. Sci. USA 1992, 89, 9367-9371; Zuckermann, R. N. et al., J. Med. Chem. 1994, 37, 2678-2685; Kruijtzer, J. A. W. & Liskamp, R. M. J. Tetrahedron Lett. 1995, 36, 6969-6972); Kruijtzer, J. A. W. Thesis; Utrecht University, 1996; Kruijtzer, J. A. W. et al., Chem. Eur. J. 1998, 4, 1570-1580), oligosulfones (Sommerfield, T. & Seebach, D. Angew. Chem., Int. Ed. Eng. 1995, 34, 553-554), phosphodiesters (Lin, P. S.; Ganesan, A. Bioorg. Med. Chem. Lett. 1998, 8, 511-514), oligosulfonamides (Moree, W. J. et al., Tetrahedron Lett. 1991, 32, 409-412; Moree, W. J. et al., Tetrahedron Lett. 1992, 33, 6389-6392; Moree, W. J. et al., Tetrahedron 1993, 49, 1133-1150; Moree, W. J. Thesis; Leiden University, 1994; Moree, W. J. et al., J. Org. Chem. 1995, 60, 5157-5169; de Bont, D. B. A. et al., Bioorg. Med. Chem. Lett. 1996, 6, 3035-3040; de Bont, D. B. A. et al., Bioorg. Med. Chem. 1996, 4, 667-672; Löwik, D. W. P. M. Thesis; Utrecht University, 1998), peptoid sulfonamides (van Ameijde, J. & Liskamp, R. M. J. Tetrahedron Lett. 2000, 41, 1103-1106), vinylogous sulfonamides (Gennari, C. et al., Eur. J. Org. Chem. 1998, 2437-2449), azatides (or hydrazinopeptides) (Han, H. & Janda, K. D. J. Am. Chem. Soc. 1996, 118, 2539-2544), oligocarbamates (Paikoff, S. J. et al., Tetrahedron Lett. 1996, 37, 5653-5656; Cho, C. Y. et al., Science 1993, 261, 1303-1305), ureapeptoids (Kruijtzer, J. A. W. et al., Tetrahedron Lett. 1997, 38, 5335-5338; Wilson, M. E. & Nowick, J. S. Tetrahedron Lett. 1998, 39, 6613-6616) and oligopyrrolinones (Smith III, A. B. et al., J. Am. Chem. Soc. 1992, 114, 10672-10674).

The vinylogous peptides and oligopyrrolinones have been developed in order to be able to form secondary structures (β-strand conformations) similar to those of peptides, or mimic secondary structures of peptides. All these oligomeric peptidomimetics are expected to be resistant to proteases and can be assembled in high-yielding coupling reactions from optically active monomers (except the peptoids).

Peptidosulfonamides are composed of α- or β-substituted amino ethane sulfonamides containing one or more sulfonamide transition-state isosteres, as an analog of the hydrolysis of the amide bond. Peptide analogs containing a transition-state analog of the hydrolysis of the amide bond have found a widespread use in the development of protease inhibitor e.g. HIV-protease inhibitors.

Another approach to develop oligomeric peptidomimetics is to completely modify the peptide backbone by replacement of all amide bonds by nonhydrolyzable surrogates e.g. carbamate, sulfone, urea and sulfonamide groups. Such oligomeric peptidomimetics may have an increased metabolic stability. Recently, an amide-based alternative oligomeric peptidomimetics has been designed viz. N-substituted Glycine-oligopeptides, the so-called peptoids. Peptoids are characterized by the presence of the amino acid side chain on the amide nitrogen as opposed to being present on the α-C-atom in a peptide, which leads to an increased metabolic stability, as well as removal of the backbone chirality. The absence of the chiral α-C atom can be considered as an advantage because spatial restrictions which are present in peptides do not exist when dealing with peptoids. Furthermore, the space between the side chain and the carbonyl group in a peptoid is identical to that in a peptide. Despite the differences between peptides and peptoids, they have been shown to give rise to biologically active compounds.

Translation of a peptide chain into a peptoid peptidomimetic may result in either a peptoid (direct-translation) or a retropeptoid (retro-sequence). In the latter category the relative orientation of the carbonyl groups to the side chains is maintained leading to a better resemblance to the parent peptide.

Review articles about peptidomimetics that are incorporated herein by reference are:
Adang, A. E. P. et al.; Recl. Trav. Chim. Pays-Bas 1994, 113, 63-78; Giannis, A. & Kolter, T. Angew. Chem. Int. Ed. Engl. 1993, 32, 1244-1267; Moos, W. H. et al., Annu. Rep. Med. Chem. 1993, 28, 315-324; Gallop, M. A. et al., J. Med. Chem. 1994, 37, 1233-1251; Olson, G. L. et al., J. Med. Chem. 1993, 36, 3039-30304; Liskamp, R. M. J. Recl. Trav. Chim. Pays-Bas 1994, 113, 1-19; Liskamp, R. M. J. Angew. Chem. Int. Ed. Engl. 1994, 33, 305-307; Gante, J. Angew. Chem. Int. Ed. Engl. 1994, 33, 1699-1720; Gordon, E. M. et al., Med. Chem. 1994, 37, 1385-1401; and Liskamp, R. M. J. Angew. Chem. Int. Ed. Engl. 1994, 33, 633-636.

The invention thus furthermore relates to molecules that are not proteins or peptides themselves but have a structure and function similar to those of the proteins described herein. Examples of such molecules are the above described peptidomimetics, but also compounds in which one or more of the amino acids are replaced by non-proteinogenic amino acids or D-amino acids. When reference is made in this application to compounds, it is intended to include also such other compounds that have a similar or the same structure and function and as a consequence a similar or the same biological activity as the proteins and peptides.

More in particular substitutions can be made with non-proteinogenic amino acids selected from the group consisting of 2-naphtylalanine (Nal(2)), β-cyclohexylalanine (Cha), p-amino-phenylalanine ((Phe(p-NH₂), p-benzoyl-phenylalanine (Bpa), ornithine (Orn), norleucine (Nle), 4-fluoro-phenylalanine (Phe(p-F)), 4-chloro-phenylalanine (Phe(p-Cl)), 4-bromo-phenylalanine (Phe(p-Br)), 4-iodo-phenylalanine (Phe(p-I)), 4-methyl-phenylalanine (Phe(p-Me)), 4-methoxy-phenylalanine (Tyr(Me)), 4-nitro-phenylalanine (Phe(p-NO2)).

Suitable D-amino acids for substituting the amino acids in the peptides of the invention are for example those that are selected from the group consisting of D-phenylalanine, D-alanine, D-arginine, D-asparagine, D-aspartic acid, D-cysteine, D-glutamic acid, D-glutamine, D-histidine, D-isoleucine, D-leucine, D-lysine, D-methionine, D-proline, D-serine, D-threonine, D-tryptophan, D-tyrosine, D-valine, D-2-naphtylalanine (D-Nal(2)), β-cyclohexyl-D-alanine (D-Cha), 4-amino-D-phenylalanine (D-Phe(p-NH₂)), p-benzoyl-D-phenylalanine (D-Bpa), D-Ornithine (D-Orn), D-Norleucine (D-Nle), 4-fluoro-D-phenylalanine (D-Phe(p-F)), 4-chloro-D-phenylalanine (D-Phe(p-Cl)), 4-bromo-D-phenylalanine (D-Phe(p-Br)), 4-iodo-D-phenylalanine (D-Phe(p-I)), 4-methyl-D-phenylalanine (D-Phe(p-Me)), 4-methoxy-D-phenylalanine (D-Tyr(Me)), 4-nitro-D-phenylalanine (D-Phe(p-NO2)).

One or more of the amino acids in the compounds of the invention can be replaced by peptoid building blocks, e.g. selected from the group consisting of N-substituted glycines, such as N-benzylglycine (NPhe), N-methylglycine (NAla), N-(3-guanidinopropyl)glycine (NArg), N-(Carboxymethyl)glycine (NAsp), N-(carbamylmethyl)glycine (NAsn), N-(thioethyl)-glycine (NhCys), N-(2-carboxyethyl)glycine (NGlu), N-(2-carbamylethyl)glycine (NGln), N-(imidazolylethyl)glycine (NhHis), N-(1-methylpropyl)glycine (NIle), N-(2-methylpropyl)glycine (NLeu), N-(4-aminobutyl)glycine (NLys), N-(2-methylthioethyl)glycine (NMet), N-(hydroxyethyl)glycine (NhSer), N-(2-hydroxypropyl)glycine (NhThr), N-(3-indolylmethyl)glycine (NTrp), N-(p-hydroxyphenmethyl)-glycine (NTyr), N-(1-methylethyl)glycine (NVal).

The present invention further relates to a pharmaceutical composition for the treatment of microbial infection, comprising a compound of the invention and a suitable excipient. The invention also relates to the use of the compounds of the invention for antimicrobial treatment and for the preparation of a pharmaceutical composition for the treatment of microbial infection.

The invention will be further illustrated in the Examples that follow and in which reference is made to the following figures.

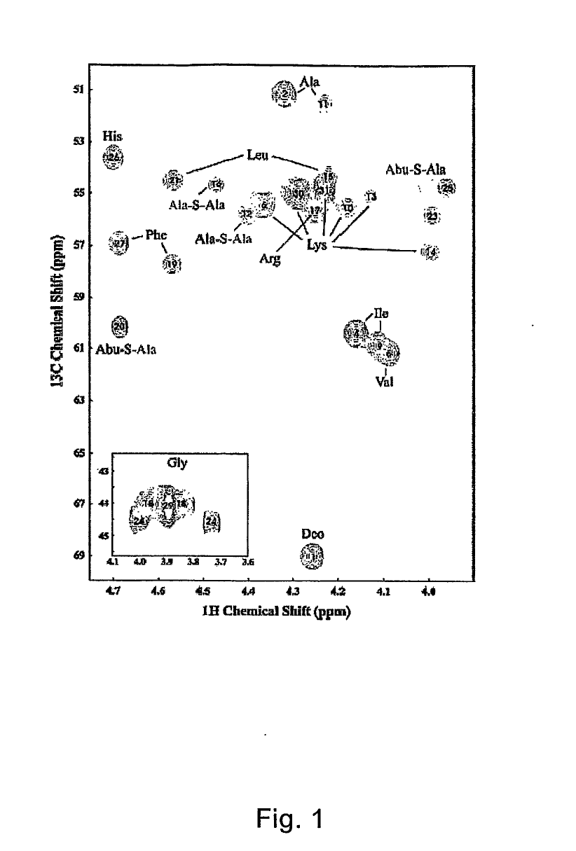

FIG. 1. The ¹H?—¹³C? region of a natural abundance ¹H—¹³C—HSQC spectrum of epilancin 15X. Each cross peak is labeled with the corresponding residue number and identity. The inset shows the region that corresponds to that of the three glycine residues identified in epilancin 15X. The structure of the post-translationally modified residues is shown on the right side of the spectrum. The N-terminus is a hydroxy-propionyl group indicated as Hop.

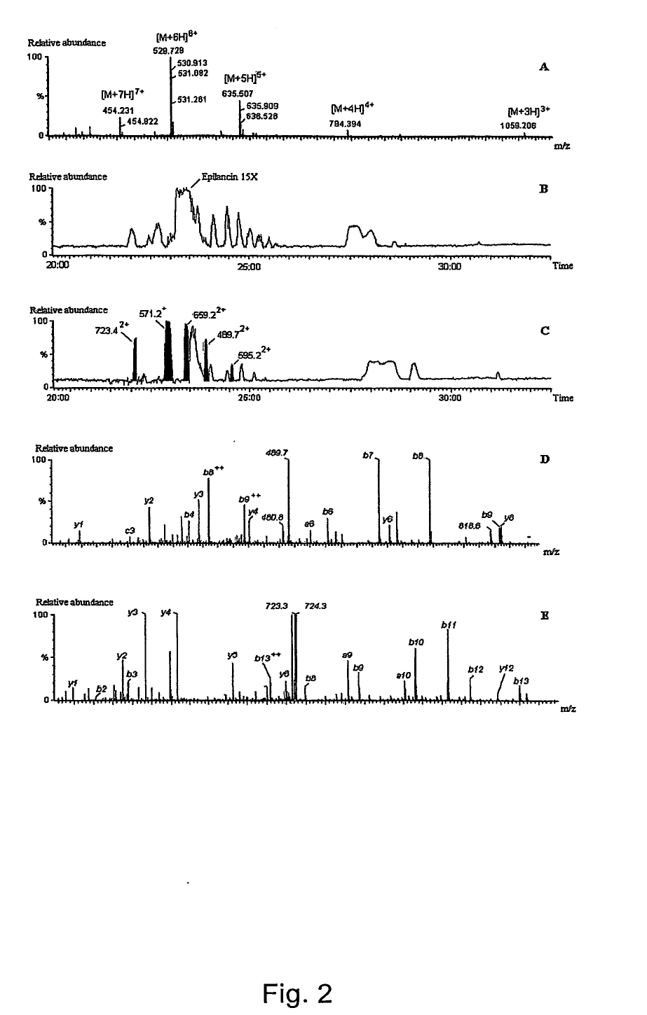

FIG. 2A displays a mass spectrum of epilancin 15X, in which the most prominent peak is the [M+6H]⁶⁺ ion. A relative abundance chromatogram of epilancin 15X is displayed in FIG. 2B and of a trypsin digest of the peptide in FIG. 2C. FIGS. 2D and 2E show tandem mass spectra of two trypsin digest fragments.

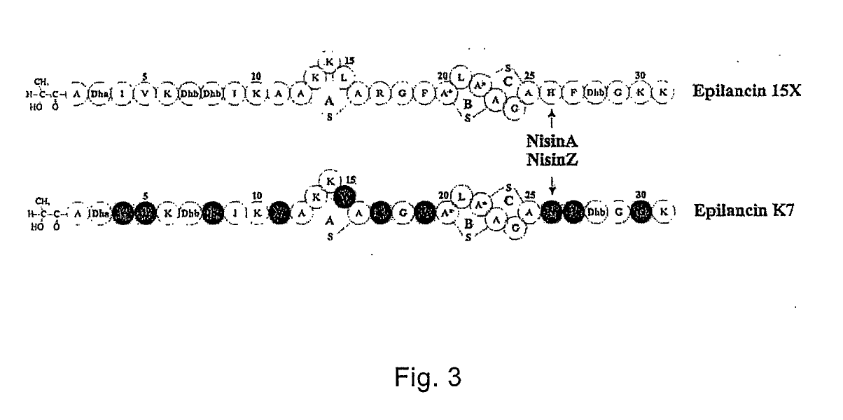

FIG. 3. Primary structure of epilancin 15X compared to that of epilancin K7. Lanthionine rings A, B and C are indicated. The residues that are different between the two lantibiotics are shaded grey. The positively charged residues are indicated by a plus sign.

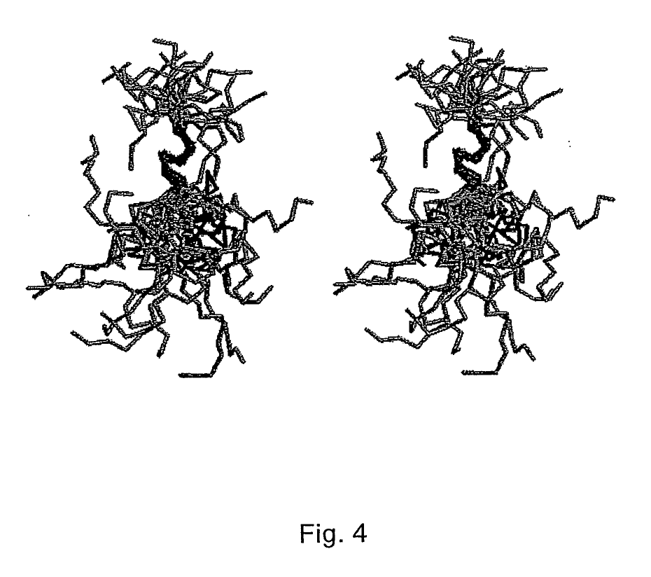

FIG. 4. The three-dimensional solution structure of epilancin 15X. A. Ensemble of 20 lowest-energy water-refined structures of epilancin 15X shown in backbone trace. Structures were superimposed on the backbone atoms (N, C?, C′) of rings B and C (residues 20-25). The N- and C-termini are indicated by the letters N and C, respectively. B. A combined ribbon/surface presentation of the lowest-energy water-refined structure of epilancin 15X. The side chains of the six lysine and single arginine residues are shown in stick representation with the corresponding residue numbers. The sulfur atoms in the three rings A, B and C are shown in spheres. The figures were generated using MolMol (Koradi, R et al., (1996) J. Mol. Graphics 14, 29-32).

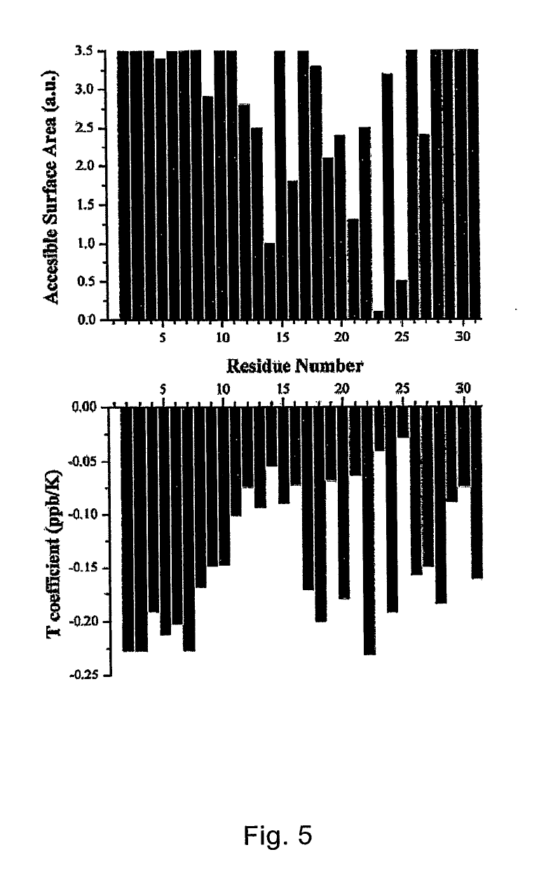

FIG. 5. NMR-derived structural data for epilancin 15X and epilancin K7. The temperature-coefficients of the individual amide protons in epilancin 15X were derived from a series of TOCSY experiments between 283 and 305 K, and are shown by dark-grey bars. The temperature-coefficients of epilancin K7 are taken from Van de Kamp et al. ((1995) Eur. J. Biochem. 230, 587-600) and are shown by white bars. Except for the first few residues, the experimentally derived temperature-coefficients for the two lantibiotics are closely similar indicating a similar 3D structure. Note the typical up-down pattern for the residues in ring B and C in both peptides. The three ring-systems A, B and C are indicated at the top of the figure.

### EXAMPLES

### Example 1

Isolation of Protein of the Invention

A single colony of Staphylococcus epidermidis strain 15X154 (CBS accession no. 113428) was inoculated in 10 ml of Mueller Hinton medium (Beckton Dickinson Diagnostic systems, Sparks, Md., USA) and incubated for 8 hours at 37° C. on a shaker at 200 rotations per minute. Subsequently, 1 litre of Mueller Hinton broth was inoculated with 200 μl of the grown culture and incubated for 16 hours at 37° C. on a shaker at 200 rotations per minute. The culture was centrifuged for 15 minutes at 9000×g and the supernatant filtrated through a 0.22 μm sterilizing filter (Millipore).

A cation exchange liquid chromatography column (SP XL 5 ml, Amersham Biosciences, Uppsala, Sweden) was equilibrated with 40 ml of 50 mM sodium phosphate buffer pH 5.0 at a flow rate of 5 ml per minute and the filtered supernatant was passed over the column at a flow rate of 5 ml per minute. The active substance was eluted in 12 minutes at a flow rate of 5 ml per minute with a linear gradient of 0 to 0.9 M sodium chloride in 50 mM sodium phosphate buffer with a pH of 5.0. 5 Ml fractions were collected and tested these according to Example 2. The active fractions were pooled and ammonium sulfate was added to a final concentration of 1.5 M for loading on the following column.

Subsequently, a hydrophobic interaction column (Source 15 PHE 4.6/100 PE, Amersham Biosciences, Uppsala, Sweden) was equilibrated with 10 ml of 1.5 M ammonium sulfate in 50 mM sodium phosphate pH 5.0 at a flowrate of 1 ml per minute and the pooled active fractions were passed over it at a flow rate of 1 ml per minute.

The active substance were eluted in 20 minutes at a flowrate of 1 ml per minute with a linear gradient of 1.5 M to 0 M ammoniumsulfate in 50 mM sodium phosphate pH 5.0. 2 Ml fractions were collected and tested according to Example 2.

The active fractions were pooled and trifluoroacetic acid was added to a final concentration of 0.1%.

Then a reverse phase liquid chromatography column (Source 15 RPC, Amersham Biosciences, Uppsala, Sweden) was equilibrate with 10 ml of methanol/0.1% trifluoroacetic acid and subsequently with 10 ml of distilled water/0.1% trifluoroacetic acid at a flow rate of 1 ml per minute. The pooled active fractions were passed over the column at a flow rate of 1 ml per minute. The active substance was eluted in 15 minutes at a flow rate of 1 ml per minute with a linear gradient of 45% methanol/55% distilled water/0.1% trifluoroacetic acid to 80% methanol/20% distilled water/0.1% acetic acid. 1 Ml fractions were collected and tested according to Example 2.

The active fractions were pooled and the methanol and trifluoroacetic acid were evaporated in a rotational vacuum concentrator until the sample contained one tenth of the original volume. Distilled water was added up to the original volume and the product was freeze-dried.

### Example 2

Activity Testing

A Mueller Hinton II agar plate (Beckton Dickinson Diagnostic systems, Sparks, Md., USA) with a diameter of 14.5 cm was dried for 2 hours in a stove at 37° C. A grid was marked with 20 evenly distributed points on the back of the agar plate. 10 μl samples of the eluted fractions were pipetted on the marked points and dried in for 20 minutes at room temperature. 5 μl of a 0.5 McFarland suspension of Staphylococcus epidermidis ATCC strain 49134 were pipetted on the same spots and the agar plate incubated in a stove for 16 hours at 37° C.

It was determined which fractions are active by identifying which samples have inhibited the growth of Staphylococcus epidermidis ATCC strain 49134.

### Example 3

Activity Assay

To assay the activity of the purified peptide against various microorganisms among which Staphylococcus aureus, Enterococcus faecalis and Acinetobacter baumanii, the freeze-dried peptide was redissolved in Mueller Hinton broth to an end concentration of 1000 μg/ml and this solution was used to make a series of twofold dilutions.

50 μl of each of these dilutions was pipetted into a separate well in a 96-well microtiter plate and 50 μl of a suspension of the bacterium to be tested for its susceptibility was added to the peptide dilutions. The bacterial suspensions tested were 0.5 McFarland suspensions which were diluted 10.000-fold.

The microtiter plate was sealed and incubated at 37° C. for 16 hours, after which it was inspected visually for inhibition of growth of the tested bacteria. Minimal inhibitory concentration (MIC) was defined as the lowest concentration of peptide at which no growth of the tested bacteria occurred.

The following tables shows the MIC for various European isolates of different bacterial species.

TABLE 1

| Species | Isolate No. | MIC (μg/ml) |
|---|---|---|
| Acinetobacter spp. | 08A568 | 25 |
| | 08A569 | 12.5 |
| | 08A608 | 12.5 |
| | 08A765 | 12.5 |
| | 08A766 | 25 |
| Escherichia coli | 05A656 | 50 |
| | 05A662 | 25 |
| | 05A663 | 50 |
| | 05A664 | 25 |
| | 05A665 | 50 |
| | 13A180 | 100 |
| | 13A181 | 100 |
| | 13A182 | 50 |
| | 13A183 | 25 |
| | 13A184 | 25 |

TABLE 2
MSSA

| | |
|---|---|
| 11a483 | 0.5 |
| 11a510 | 0.5 |
| 13a214 | 0.25 |
| 13a217 | 0.25 |
| 13a222 | 0.25 |
| 15a691 | 0.5 |
| 15a701 | 0.5 |
| 18a701 | 0.25 |
| 18a706 | 0.25 |
| ATCC29213 | 0.5 |

MRSA

| | |
|---|---|
| 02a042 | 0.25 |
| 05c013 | <0.125 |
| 06a027 | 0.25 |
| 07c007 | 0.25 |
| 10d005 | 0.5 |
| 11a139 | 0.5 |
| 11a251 | 0.25 |
| 16a517 | 0.25 |

Enterococcus faecium

| | |
|---|---|
| 07a727 | 0.25 |
| 08a849 | 0.5 |
| a: 7E116 | 0.5 |
| 15a800 | 0.25 |
| 15a801 | 0.25 |

Enterococcus faecalis

| | |
|---|---|
| 08a842 | 0.25 |
| 13a266 | 0.5 |
| a: 15e209 | 0.5 |
| a: 15E207 | 0.5 |
| 18a676 | 1.0 |
| ATCC 29212 | 0.5 |

VRE

| | |
|---|---|
| 13a248 | <0.125 |
| 15a693 | 0.25 |
| 15a794 | <0.125 |
| 15a799 | 0.25 |
| a: 15e179 | 0.5 |

S. epidermidis

| | |
|---|---|
| 05a759 | <0.125 |
| 05a760 | 0.25 |
| a: 09a351 | <0.125 |
| 18a613 | <0.125 |
| 18a626 | <0.125 |
| 18a630 | <0.125 |
| 18a632 | <0.125 |
| 18a643 | <0.125 |
| 30a288 | <0.125 |
| 15x154 | 1.0 |

### Example 3

Isolation and Purification of Epilancin 15X

Materials and Methods

Isolation and Purification

A 1.5-liter Mueller Hinton broth (MH-b) culture was inoculated with 100 μl of overnight S. epidermidis 15X154 culture and grown for 16 h at 37° C. on a shaker. After centrifugation for 15 minutes at 9000 rpm, the supernatant was filter sterilized and purified on an Äkta FPLC system (Amersham, Uppsala, Sweden) by cation exchange chromatography (HiTrap SP XL), hydrophobic interaction chromatography (Source 15 PHE) and reverse phase chromatography (Source 15 RPC, all chromatography columns purchased from Amersham, Uppsala, Sweden). The purified compound was stored at 4° C., either dissolved in water or lyophilized.

Mass Spectrometry

Nano-scale liquid chromatography mass spectrometry (nLC-MS) and tandem mass spectrometry (nLC-MS2) were performed on a 1100 series liquid chromatography system (Agilent Technologies, Palo Alto, Calif.) and a Q-T of Ultima API hybrid quadrupole/time of flight mass spectrometer (Waters corporation, Milford, Mass.) as described previously by Meiring et al. ((2002) J. Sep. Science 25, 557-568). Samples were diluted to 1 fmol/μl in 5% DMSO/5% formic acid and 10 μl was injected onto a biosphere C18 trapping column (20 mm, 100 μm diameter) with a 125 nanoliter/min flowrate. Analyte separation was performed on a biosphere C18 column (25 cm, 50 μm diameter), with a linear gradient in 20 minutes from 0 to 60% acetonitril with 0.1 M acetic acid, at a flowrate of 3 μl/minute (both columns manufactured by Nanoseparations, Nieuwkoop, The Netherlands). Trapping column, analytical column and electrospray ionization (ESI) tip were butt-connected to one another, hereby providing a near to zero dead volume. Data were analyzed using the MassLynx 3.4 software.

Peptide Digestion

The peptide was hydrolyzed with modified trypsin according to the procedure provided by the supplier (Boehringer Mannheim, Germany). Approximately 100 pmol of peptide was incubated with 20 pmol enzyme in a buffer solution (100 μl) for 4 h at 37° C. Digestion was terminated by acidification with 1 μl of acetic acid.

NMR Spectroscopy

¹H homonuclear NMR experiments were carried out with 1.25 mg of epilancin 15X dissolved in 500 ml 90% H₂O/10% D₂O, 10 mM d3-sodium acetate at pH 4. Heteronuclear experiments were carried out with 3 mg epilancin 15X in 300 ml D2O using a shigemi tube. 2D ¹H total correlation spectroscopy (TOCSY) and 2D ¹H nuclear Overhauser enhancement spectroscopy (NOESY) spectra were recorded on a Bruker DRX 750 MHz spectrometer, both at 305 K and 283 K using spectral widths of 12 ppm in both dimensions. NOESY spectra were collected using mixing times of 100 ms, 300 ms and 500 ms to check for spin diffusion effects. The mixing time for the TOCSY spectra was 80 ms using the DIPSI-2 pulsetrain for spin-lock. Suppression of the water signal was achieved by a weak on-resonance iradiation during the 2 s relaxation time and WATERGATE (Piotto, M et al., (1992) J. Biomol. NMR 2, 661-665) for samples in D₂O and water, respectively.

Natural abundance ¹H—¹³C hetero-nuclear single quantum correlation (HSQC) and ¹H—¹³C hetero-nuclear multiple bond correlation (HMBC) NMR spectra were acquired on a Bruker Avance 600 MHz spectrometer equipped with a cryoprobe system at 283 K. The spectra were acquired with 1K×400 points with 80 scans and 256 scans per increment, respectively. The ¹H spectral width was 10 ppm in both experiments, and the ¹³C spectral widths were 150 and 200 ppm, respectively. Both dimensions were zero-filled to yield 2K×1K real points. For the HSQC a 900 phase shifted square sine-bell window function was applied in both dimensions before Fourier transformation. For the HMBC an exponential multiplication was applied in the direct dimension and a 600 phase shifted square sine-bell window function was applied in the indirect dimension before Fourier transformation. All spectra were processed using the NMRPipe software package (Delaglio, F et al. (1995) J. Biomol. NMR 6, 277-293) and analyzed using NMRView (Johnson, B. A. and Blevins, R. A. (1994) J. Biomol. NMR 4, 603-614).

Structure Calculation and Analysis

All structure calculations were performed with CNS (Brünger, A. T. et al., (1998) Acta Crystallogr. D Biol. Crystallogr. 54 (Pt 5), 905-921) using the ARIA setup and protocols (Nilges, M. and Donoghue, S. I. (1998) Prog. Nucl. Magn. Reson. Spectrosc. 32, 107-139). Most peaks in the NOESY spectra were unambiguously assigned, except those for those that show spectral overlap, which were assigned as ambiguous with a lower weighing factor. Distance restraints were derived from NOESY peak intensities. The calibration of the cross-peak intensities against distances was done automatically at the beginning of each iteration. Each semi-automated assignment step with ARIA consisted of eight iterations with successive reduction of the violation tolerance and a final refinement in explicit solvent using default ARIA parameters. The modified Parallhdg5.3.1 force field with the PROLSQ parameters (Linge, J. P. et al., (2003) Proteins 50, 496-506) and additional definitions for dehydroalanine (Dha), dehydrobutirine (Dhb), 3-methyllanthionine (MeLan) and lanthione (Lan) (Hsu, S.-T. D. et al., (2003) J. Biol. Chem. 278, 13110-13117) were used. The topology for hydroxy-propionyl (Hop) was constructed based on alanine and with comparison of available databases. Three thio-ether bridges were introduced. A torsion angle dynamics (TAD) simulated annealing (SA) protocol was performed, initially at 10000 K (10000 steps), followed by a first cooling stage to 50 K (50K/step); Cartesian space refinement was used for the second cooling stage (from 2000K to 1000 K in 5000 steps) and the subsequent third cooling stage (from 1000 K to 50 K in 10000 steps) followed by 200 steps of energy minimization. The slow cooling process at the second stage ensures a better convergence of the calculated structures. All 100 structures were further subjected to explicit solvent refinement (Linge et al. (2003), supra) and the 20 lowest-energy structures were kept for structural analysis.

Results and Discussion

Isolation and Purification

Pure epilancin 15X was obtained in a three-step liquid chromatography setup. The product could be isolated from grown cultures in about eight hours. The yield was highest after a culture incubation of twelve to sixteen hours, in late log-phase/early stationary phase, and was typically 0.5 mg peptide/litre culture.

Spin System Analysis & Sequence Determination by NMR

The number and nature of residues of the peptide was determined using the 2D ¹H TOCSY and 2D ¹H NOESY spectra recorded at 283 K. No signs of spin diffusion were observed in the 300 ms NOESY spectrum. For an initial identification of each spin system, chemical shifts were compared with random coil values (Wishart, D. S. and Nip, A. M. (1998) Biochem. Cell Biol. 76, 153-163). A similar approach was possible for the modified amino acids, by comparing their chemical shifts with the chemical shift values of such residues in other lantibiotics (van de Kamp, M. et al., (1995) Eur. J. Biochem. 227, 757-771; Chan, W. C. et al., (1992) FEBS Lett. 300, 56-62; Chan, W. C. et al., (1989) J. Chem. Soc. Perkin. Trans. I, 2359-2367; Kuipers, O. P. et al., (1992) J. Biol. Chem. 267, 24340-24346).

Sequential assignment was achieved using standard NOE connectivity-based protocol (Wüthrich, K. (1986) NMR of Proteins and Nucleic Acids, John Wiley and Sons, New York). Problems of spectral overlap were resolved by inspection of either homonuclear NMR spectra recorded at higher temperature (305 K) or by the ¹³C heteronuclear spectra. All residues could be identified and sequentially assigned in this way.

The results were then confirmed by the ¹H—¹³C HMBC experiment. In addition, the combined use of the ¹H—¹³C HSQC and ¹H—¹³C HMBC spectra enabled almost complete ¹³C chemical shift assignment of the side chain carbon atoms and backbone carbonyls.

FIG. 1 shows the ¹H?—¹³C? region of the natural abundance ¹H—¹³C HSQC with the assignments obtained following the procedure described above.

During the assignment process, a peak was noted originating from the N-terminal amide proton that is usually not visible by NMR spectroscopy due to its fast exchange with water. This lead to the assumption that a yet unassigned chemical entity is present preceding the alanine residue, which was first presumed to represent the N-terminus. After close inspection of all NMR spectra recorded in conjunction with the molecular weight obtained from mass spectrometry (see the following section) the N-terminus could be unambiguously assigned to Hop (FIG. 1).

The combined analysis of the TOCSY, NOESY, HSQC and HMBC spectra thus resulted in the identification of the primary sequence of this molecule with a total number of 31 residues, including ten modified amino acids, three lanthionine ring structures (denoted A, B and C in FIG. 3), and a hydroxy-propionyl N-terminal moiety. The presence and nature of the lanthionine rings could be confirmed by the NOE connectivities within the residue pairs that are involved in the lanthionine linkages.

Determination of the Mass Using Mass Spectrometry

The NMR sequence determination described above revealed a lantibiotic containing 31 residues with a calculated mass of 3173 Da. In order to verify the sequence thus determined, mass spectra of digested and undigested peptide samples were recorded by nLC-MS and relative abundance chromatograms were compared (FIG. 2A and FIG. 2B). The mass spectrum of the undigested peptide (FIG. 2C) yielded ions in charge states ranging from 3⁺ to 7⁺, the [M+6H]⁶⁺ ions with a m/z of 529.7 being the most abundant. A molecular weight of 3172.9 Da was calculated in agreement with the mass determined from the NMR derived sequence. A nLC-MS² spectrum of the [M+4H]⁴⁺ ion, with m/z of 794.2 was recorded and averaged for analysis.

In the relative abundance chromatograms of a peptide digest of the molecule, five new peaks could be distinguished: 723.3²⁺, 571.2⁺, 659.1²⁺, 489.7²⁺ and 595.2²⁺. These ions were selected for recording and analysis of nLC-MS2 spectra and were found to correspond to residues 1-5, 1-10 (FIG. 2D), 18-29, 18-30 and 18-31 (FIG. 2E). No digestion fragment corresponding to residues 11-17 could be identified. The expected m/z ratio of this fragment (calculated size 787 Da) with three positively charged amino acids would be 263.3 for the [M+3H]³⁺ ion and 197.7 for the [M+4H]⁴⁺ ion, and fallS outside the range (m/z 300-2000) of the quadrupole mass spectrometer. Presence of Ala11 could be confirmed by the presence of y20 and y21-ions in the nLC-MS² spectrum of the undigested peptide and the calculated size of the Ala12-Arg17 fragment (697.3 Da) corresponded with the difference between the y20-ion and the y12-ion, minus the measured size of Gly18-Phe19.

In general, b-ions and y-ion were the most prominent, but in the mass spectra of the digest fragments also series of a-ions could be identified. Y-ions were often accompanied by peaks of 18 Da smaller, attributable to loss of H₂O-groups. The nLC-MS² spectrum of the 1-5 digest fragment displayed a number of peaks 14 Da smaller than (predicted) a- and b-ions (namely: 284.2; 312.2; 383.3; 411.2), which can be explained by loss of a CH₂ group by the hydroxy-propionyl residue.

Little cleavage occurred between residues Abu20 and Ala25, a part of the molecule in which two β-methyllanthionine rings are present. Masses of 288.1 and 401.2 were found in the spectra of all three digest fragments 18-29, 18-30 and 18-31 (and therefore necessarily N-terminal corresponding to the b3 and b4-ions formed by cleavage between Abu20 and Leu21, and between Leu21 and Abu22. The mass difference of 83 Da between the b2 (205.1) and b3-ion suggests loss of a hydrogen atom and formation of Dhb from Abu after cleavage of the thioether bridge.

FIG. 3 shows the primary sequence of the novel lantibiotic obtained from the combined use of NMR and mass spectrometry. There is an homology of 68% between the sequence thus determined and that of eplinacin K7. Epilancin 15X contains seven positively charged residues (six lysines, one arginine) and three ring structures (A, B and C in FIG. 3). The cleavage sites retrieved by nLC-MS2 are also indicated in the figure.

The 3D Solution Structure of Epilancin 15X and Comparison with Epilancin K7

The solution structure of epilancin 15X was determined based on the NOE-derived distance restraints (Table 3). Like many type A lantibiotics, it exhibits no conventional secondary structure but well-defined local ring structures (FIG. 4 and Table 3). In order to validate the structures calculated for epilancin 15X, the temperature-coefficients of the individual amide protons was measured and compared with those measured for epilancin K7 (Van de Kamp, M. et al (1995), supra). FIG. 5 shows the values for both lantibiotics.

TABLE 3
Structural statistics for an ensemble of twenty lowestenergy
water-refined structures of Epilancin 15X

| Distance restraints¹ | |
|---|---|
| Intraresidue (i − j = 0) | 209 |
| Sequential (\|i − j\| = 1) | 92 |
| Medium range (2 ≦ \|i − j\| =≦ 4) | 30 |
| All | 331 |
| Restraint statistics | |
| NOE RMSD (10⁻² Å) | 2.93 +/− 0.26 |
| violations >0.5 Å | 0 |
| violations >0.3 Å | 0.05 +/− 0.22 |
| violations >0.1 Å | 2.6 +/− 6.5 |
| RMSD from idealized covalent | |
| geometry | |
| Bonds (Å) | 0.0034 +/− 0.00019 |
| Angles (°) | 0.580 +/− 0.025 |
| Impropers (°) | 1.27 +/− 0.15 |
| Dihedrals (°) | 42.6 +/− 1.4 |
| CNS energies (kcal/mol) | |
| Eₜₒₜₐₗ | −806.4 +/− 36.5 |
| E_{vdw} | −170.1 +/− 4.6 |
| E_{elec} | −886.01 +/− 35.6 |
| Backbone (N, CA, C′) pairwise | |
| RMSD2 in Å | |
| All residues | 7.63 +/− 2.14 |
| Residues 12-16 (ring A) | 1.01 +/− 0.28 |
| Residues 20-25 (ring B + C) | 0.61 +/− 0.34 |

¹Distance restraint statistics reported for unique, unambiguous assigned NOEs.
²Coordinate precision is given as the average pairwise Cartesian coordinate Root Mean Square Deviations over the ensemble.

TABLE 4
All combinations of substituted positions in relation to K7

4-5-8-11-15-17-19-26-27
4-5-8-11-15-17-19-26-30
4-5-8-11-15-17-19-26-27-30
4-5-8-11-15-17-19-27
4-5-8-11-15-17-19-30
4-5-8-11-15-17-19-27-30
4-5-8-11-15-17-26-27
4-5-8-11-15-17-26-30
4-5-8-11-15-17-26-27-30
4-5-8-11-15-17-27
4-5-8-11-15-17-30
4-5-8-11-15-17-27-30
4-5-8-11-15-19-26-27
4-5-8-11-15-19-26-30
4-5-8-11-15-19-26-27-30
4-5-8-11-15-19-27
4-5-8-11-15-19-30
4-5-8-11-15-19-27-30
4-5-8-11-15-26-27
4-5-8-11-15-26-30
4-5-8-11-15-26-27-30
4-5-8-11-15-27
4-5-8-11-15-30
4-5-8-11-15-27-30
4-5-8-11-17-19-26-27
4-5-8-11-17-19-26-30
4-5-8-11-17-19-26-27-30
4-5-8-11-17-19-27
4-5-8-11-17-19-30
4-5-8-11-17-19-27-30
4-5-8-11-17-26-27
4-5-8-11-17-26-30
4-5-8-11-17-26-27-30
4-5-8-11-17-27
4-5-8-11-17-30
4-5-8-11-17-27-30
4-5-8-11-19-26-27
4-5-8-11-19-26-30
4-5-8-11-19-26-27-30
4-5-8-11-19-27
4-5-8-11-19-30
4-5-8-11-19-27-30
4-5-8-11-26-27
4-5-8-11-26-30
4-5-8-11-26-27-30
4-5-8-11-27
4-5-8-11-30
4-5-8-11-27-30
4-5-8-15-17-19-26-27
4-5-8-15-17-19-26-30
4-5-8-15-17-19-26-27-30
4-5-8-15-17-19-27
4-5-8-15-17-19-30
4-5-8-15-17-19-27-30
4-5-8-15-17-26-27
4-5-8-15-17-26-30
4-5-8-15-17-26-27-30
4-5-8-15-17-27
4-5-8-15-17-30
4-5-8-15-17-27-30
4-5-8-15-19-26-27
4-5-8-15-19-26-30
4-5-8-15-19-26-27-30
4-5-8-15-19-27
4-5-8-15-19-30
4-5-8-15-19-27-30
4-5-8-15-26-27
4-5-8-15-26-30
4-5-8-15-26-27-30
4-5-8-15-27
4-5-8-15-30
4-5-8-15-27-30
4-5-8-17-19-26-27
4-5-8-17-19-26-30
4-5-8-17-19-26-27-30
4-5-8-17-19-27
4-5-8-17-19-30
4-5-8-17-19-27-30
4-5-8-17-26-27
4-5-8-17-26-30
4-5-8-17-26-27-30
4-5-8-17-27
4-5-8-17-30
4-5-8-17-27-30
4-5-8-19-26-27
4-5-8-19-26-30
4-5-8-19-26-27-30
4-5-8-19-27
4-5-8-19-30
4-5-8-19-27-30
4-5-8-26-27
4-5-8-26-30
4-5-8-26-27-30
4-5-8-27
4-5-8-30
4-5-8-27-30
4-5-11-15-17-19-26-27
4-5-11-15-17-19-26-30
4-5-11-15-17-19-26-27-30
4-5-11-15-17-19-27
4-5-11-15-17-19-30
4-5-11-15-17-19-27-30
4-5-11-15-17-26-27
4-5-11-15-17-26-30
4-5-11-15-17-26-27-30
4-5-11-15-17-27
4-5-11-15-17-30
4-5-11-15-17-27-30
4-5-11-15-19-26-27
4-5-11-15-19-26-30
4-5-11-15-19-26-27-30
4-5-11-15-19-27
4-5-11-15-19-30
4-5-11-15-19-27-30
4-5-11-15-26-27
4-5-11-15-26-30
4-5-11-15-26-27-30
4-5-11-15-27
4-5-11-15-30
4-5-11-15-27-30
4-5-11-17-19-26-27
4-5-11-17-19-26-30
4-5-11-17-19-26-27-30
4-5-11-17-19-27
4-5-11-17-19-30
4-5-11-17-19-27-30
4-5-11-17-26-27
4-5-11-17-26-30
4-5-11-17-26-27-30
4-5-11-17-27
4-5-11-17-30
4-5-11-17-27-30
4-5-11-19-26-27
4-5-11-19-26-30
4-5-11-19-26-27-30
4-5-11-19-27
4-5-11-19-30
4-5-11-19-27-30
4-5-11-26-27
4-5-11-26-30
4-5-11-26-27-30
4-5-11-27
4-5-11-30
4-5-11-27-30
4-5-15-17-19-26-27
4-5-15-17-19-26-30
4-5-15-17-19-26-27-30
4-5-15-17-19-27
4-5-15-17-19-30
4-5-15-17-19-27-30
4-5-15-17-26-27
4-5-15-17-26-30
4-5-15-17-26-27-30
4-5-15-17-27
4-5-15-17-30
4-5-15-17-27-30
4-5-15-19-26-27
4-5-15-19-26-30
4-5-15-19-26-27-30
4-5-15-19-27
4-5-15-19-30
4-5-15-19-27-30
4-5-15-26-27
4-5-15-26-30
4-5-15-26-27-30
4-5-15-27
4-5-15-30
4-5-15-27-30
4-5-17-19-26-27
4-5-17-19-26-30
4-5-17-19-26-27-30
4-5-17-19-27
4-5-17-19-30
4-5-17-19-27-30
4-5-17-26-27
4-5-17-26-30
4-5-17-26-27-30
4-5-17-27
4-5-17-30
4-5-17-27-30
4-5-19-26-27
4-5-19-26-30
4-5-19-26-27-30
4-5-19-27
4-5-19-30
4-5-19-27-30
4-5-26-27
4-5-26-30
4-5-26-27-30
4-5-27
4-5-30
4-5-27-30
4-8-11-15-17-19-26-27
4-8-11-15-17-19-26-30
4-8-11-15-17-19-26-27-30
4-8-11-15-17-19-27
4-8-11-15-17-19-30
4-8-11-15-17-19-27-30
4-8-11-15-17-26-27
4-8-11-15-17-26-30
4-8-11-15-17-26-27-30
4-8-11-15-17-27
4-8-11-15-17-30
4-8-11-15-17-27-30
4-8-11-15-19-26-27
4-8-11-15-19-26-30
4-8-11-15-19-26-27-30
4-8-11-15-19-27
4-8-11-15-19-30
4-8-11-15-19-27-30
4-8-11-15-26-27
4-8-11-15-26-30
4-8-11-15-26-27-30
4-8-11-15-27
4-8-11-15-30
4-8-11-15-27-30
4-8-11-17-19-26-27
4-8-11-17-19-26-30
4-8-11-17-19-26-27-30
4-8-11-17-19-27
4-8-11-17-19-30
4-8-11-17-19-27-30
4-8-11-17-26-27
4-8-11-17-26-30
4-8-11-17-26-27-30
4-8-11-17-27
4-8-11-17-30
4-8-11-17-27-30
4-8-11-19-26-27
4-8-11-19-26-30
4-8-11-19-26-27-30
4-8-11-19-27
4-8-11-19-30
4-8-11-19-27-30
4-8-11-26-27
4-8-11-26-30
4-8-11-26-27-30
4-8-11-27
4-8-11-30
4-8-11-27-30
4-8-15-17-19-26-27
4-8-15-17-19-26-30
4-8-15-17-19-26-27-30
4-8-15-17-19-27
4-8-15-17-19-30
4-8-15-17-19-27-30
4-8-15-17-26-27
4-8-15-17-26-30
4-8-15-17-26-27-30
4-8-15-17-27
4-8-15-17-30
4-8-15-17-27-30
4-8-15-19-26-27
4-8-15-19-26-30
4-8-15-19-26-27-30
4-8-15-19-27
4-8-15-19-30
4-8-15-19-27-30
4-8-15-26-27
4-8-15-26-30
4-8-15-26-27-30
4-8-15-27
4-8-15-30
4-8-15-27-30
4-8-17-19-26-27
4-8-17-19-26-30
4-8-17-19-26-27-30
4-8-17-19-27
4-8-17-19-30
4-8-17-19-27-30
4-8-17-26-27
4-8-17-26-30
4-8-17-26-27-30
4-8-17-27
4-8-17-30
4-8-17-27-30
4-8-19-26-27
4-8-19-26-30
4-8-19-26-27-30
4-8-19-27
4-8-19-30
4-8-19-27-30
4-8-26-27
4-8-26-30
4-8-26-27-30
4-8-27
4-8-30
4-8-27-30
4-11-15-17-19-26-27
4-11-15-17-19-26-30
4-11-15-17-19-26-27-30
4-11-15-17-19-27
4-11-15-17-19-30
4-11-15-17-19-27-30
4-11-15-17-26-27
4-11-15-17-26-30
4-11-15-17-26-27-30
4-11-15-17-27
4-11-15-17-30
4-11-15-17-27-30
4-11-15-19-26-27
4-11-15-19-26-30
4-11-15-19-26-27-30
4-11-15-19-27
4-11-15-19-30
4-11-15-19-27-30
4-11-15-26-27
4-11-15-26-30
4-11-15-26-27-30
4-11-15-27
4-11-15-30
4-11-15-27-30
4-11-17-19-26-27
4-11-17-19-26-30
4-11-17-19-26-27-30
4-11-17-19-27
4-11-17-19-30
4-11-17-19-27-30
4-11-17-26-27
4-11-17-26-30
4-11-17-26-27-30
4-11-17-27
4-11-17-30
4-11-17-27-30
4-11-19-26-27
4-11-19-26-30
4-11-19-26-27-30
4-11-19-27
4-11-19-30
4-11-19-27-30
4-11-26-27
4-11-26-30
4-11-26-27-30
4-11-27
4-11-30
4-11-27-30
4-15-17-19-26-27
4-15-17-19-26-30
4-15-17-19-26-27-30
4-15-17-19-27
4-15-17-19-30
4-15-17-19-27-30
4-15-17-26-27
4-15-17-26-30
4-15-17-26-27-30
4-15-17-27
4-15-17-30
4-15-17-27-30
4-15-19-26-27
4-15-19-26-30
4-15-19-26-27-30
4-15-19-27
4-15-19-30
4-15-19-27-30
4-15-26-27
4-15-26-30
4-15-26-27-30
4-15-27
4-15-30
4-15-27-30
4-17-19-26-27
4-17-19-26-30
4-17-19-26-27-30
4-17-19-27
4-17-19-30
4-17-19-27-30
4-17-26-27
4-17-26-30
4-17-26-27-30
4-17-27
4-17-30
4-17-27-30
4-19-26-27
4-19-26-30
4-19-26-27-30
4-19-27
4-19-30
4-19-27-30
4-26-27
4-26-30
4-26-27-30
4-27
4-30
4-27-30
5-8-11-15-17-19-26-27
5-8-11-15-17-19-26-30
5-8-11-15-17-19-26-27-30
5-8-11-15-17-19-27
5-8-11-15-17-19-30
5-8-11-15-17-19-27-30
5-8-11-15-17-26-27
5-8-11-15-17-26-30
5-8-11-15-17-26-27-30
5-8-11-15-17-27
5-8-11-15-17-30
5-8-11-15-17-27-30
5-8-11-15-19-26-27
5-8-11-15-19-26-30
5-8-11-15-19-26-27-30
5-8-11-15-19-27
5-8-11-15-19-30
5-8-11-15-19-27-30
5-8-11-15-26-27
5-8-11-15-26-30
5-8-11-15-26-27-30
5-8-11-15-27
5-8-11-15-30
5-8-11-15-27-30
5-8-11-17-19-26-27
5-8-11-17-19-26-30
5-8-11-17-19-26-27-30
5-8-11-17-19-27
5-8-11-17-19-30
5-8-11-17-19-27-30
5-8-11-17-26-27
5-8-11-17-26-30
5-8-11-17-26-27-30
5-8-11-17-27
5-8-11-17-30
5-8-11-17-27-30
5-8-11-19-26-27
5-8-11-19-26-30
5-8-11-19-26-27-30
5-8-11-19-27
5-8-11-19-30
5-8-11-19-27-30
5-8-11-26-27
5-8-11-26-30
5-8-11-26-27-30
5-8-11-27
5-8-11-30
5-8-11-27-30
5-8-15-17-19-26-27
5-8-15-17-19-26-30
5-8-15-17-19-26-27-30
5-8-15-17-19-27
5-8-15-17-19-30
5-8-15-17-19-27-30
5-8-15-17-26-27
5-8-15-17-26-30
5-8-15-17-26-27-30
5-8-15-17-27
5-8-15-17-30
5-8-15-17-27-30
5-8-15-19-26-27
5-8-15-19-26-30
5-8-15-19-26-27-30
5-8-15-19-27
5-8-15-19-30
5-8-15-19-27-30
5-8-15-26-27
5-8-15-26-30
5-8-15-26-27-30
5-8-15-27
5-8-15-30
5-8-15-27-30
5-8-17-19-26-27
5-8-17-19-26-30
5-8-17-19-26-27-30
5-8-17-19-27
5-8-17-19-30
5-8-17-19-27-30
5-8-17-26-27
5-8-17-26-30
5-8-17-26-27-30
5-8-17-27
5-8-17-30
5-8-17-27-30
5-8-19-26-27
5-8-19-26-30
5-8-19-26-27-30
5-8-19-27
5-8-19-30
5-8-19-27-30
5-8-26-27
5-8-26-30
5-8-26-27-30
5-8-27
5-8-30
5-8-27-30
5-11-15-17-19-26-27
5-11-15-17-19-26-30
5-11-15-17-19-26-27-30
5-11-15-17-19-27
5-11-15-17-19-30
5-11-15-17-19-27-30
5-11-15-17-26-27
5-11-15-17-26-30
5-11-15-17-26-27-30
5-11-15-17-27
5-11-15-17-30
5-11-15-17-27-30
5-11-15-19-26-27
5-11-15-19-26-30
5-11-15-19-26-27-30
5-11-15-19-27
5-11-15-19-30
5-11-15-19-27-30
5-11-15-26-27
5-11-15-26-30
5-11-15-26-27-30
5-11-15-27
5-11-15-30
5-11-15-27-30
5-11-17-19-26-27
5-11-17-19-26-30
5-11-17-19-26-27-30
5-11-17-19-27
5-11-17-19-30
5-11-17-19-27-30
5-11-17-26-27
5-11-17-26-30
5-11-17-26-27-30
5-11-17-27
5-11-17-30
5-11-17-27-30
5-11-19-26-27
5-11-19-26-30
5-11-19-26-27-30
5-11-19-27
5-11-19-30
5-11-19-27-30
5-11-26-27
5-11-26-30
5-11-26-27-30
5-11-27
5-11-30
5-11-27-30
5-15-17-19-26-27
5-15-17-19-26-30
5-15-17-19-26-27-30
5-15-17-19-27
5-15-17-19-30
5-15-17-19-27-30
5-15-17-26-27
5-15-17-26-30
5-15-17-26-27-30
5-15-17-27
5-15-17-30
5-15-17-27-30
5-15-19-26-27
5-15-19-26-30
5-15-19-26-27-30
5-15-19-27
5-15-19-30
5-15-19-27-30
5-15-26-27
5-15-26-30
5-15-26-27-30
5-15-27
5-15-30
5-15-27-30
5-17-19-26-27
5-17-19-26-30
5-17-19-26-27-30
5-17-19-27
5-17-19-30
5-17-19-27-30
5-17-26-27
5-17-26-30
5-17-26-27-30
5-17-27
5-17-30
5-17-27-30
5-19-26-27
5-19-26-30
5-19-26-27-30
5-19-27
5-19-30
5-19-27-30
5-26-27
5-26-30
5-26-27-30
5-27
5-30
5-27-30
8-11-15-17-19-26-27
8-11-15-17-19-26-30
8-11-15-17-19-26-27-30
8-11-15-17-19-27
8-11-15-17-19-30
8-11-15-17-19-27-30
8-11-15-17-26-27
8-11-15-17-26-30
8-11-15-17-26-27-30
8-11-15-17-27
8-11-15-17-30
8-11-15-17-27-30
8-11-15-19-26-27
8-11-15-19-26-30
8-11-15-19-26-27-30
8-11-15-19-27
8-11-15-19-30
8-11-15-19-27-30
8-11-15-26-27
8-11-15-26-30
8-11-15-26-27-30
8-11-15-27
8-11-15-30
8-11-15-27-30
8-11-17-19-26-27
8-11-17-19-26-30
8-11-17-19-26-27-30
8-11-17-19-27
8-11-17-19-30
8-11-17-19-27-30
8-11-17-26-27
8-11-17-26-30
8-11-17-26-27-30
8-11-17-27
8-11-17-30
8-11-17-27-30
8-11-19-26-27
8-11-19-26-30
8-11-19-26-27-30
8-11-19-27
8-11-19-30
8-11-19-27-30
8-11-26-27
8-11-26-30
8-11-26-27-30
8-11-27
8-11-30
8-11-27-30
8-15-17-19-26-27
8-15-17-19-26-30
8-15-17-19-26-27-30
8-15-17-19-27
8-15-17-19-30
8-15-17-19-27-30
8-15-17-26-27
8-15-17-26-30
8-15-17-26-27-30
8-15-17-27
8-15-17-30
8-15-17-27-30
8-15-19-26-27
8-15-19-26-30
8-15-19-26-27-30
8-15-19-27
8-15-19-30
8-15-19-27-30
8-15-26-27
8-15-26-30
8-15-26-27-30
8-15-27
8-15-30
8-15-27-30
8-17-19-26-27
8-17-19-26-30
8-17-19-26-27-30
8-17-19-27
8-17-19-30
8-17-19-27-30
8-17-26-27
8-17-26-30
8-17-26-27-30
8-17-27
8-17-30
8-17-27-30
8-19-26-27
8-19-26-30
8-19-26-27-30
8-19-27
8-19-30
8-19-27-30
8-26-27
8-26-30
8-26-27-30
8-27
8-30
8-27-30
11-15-17-19-26-27
11-15-17-19-26-30
11-15-17-19-26-27-30
11-15-17-19-27
11-15-17-19-30
11-15-17-19-27-30
11-15-17-26-27
11-15-17-26-30
11-15-17-26-27-30
11-15-17-27
11-15-17-30
11-15-17-27-30
11-15-19-26-27
11-15-19-26-30
11-15-19-26-27-30
11-15-19-27
11-15-19-30
11-15-19-27-30
11-15-26-27
11-15-26-30
11-15-26-27-30
11-15-27
11-15-30
11-15-27-30
11-17-19-26-27
11-17-19-26-30
11-17-19-26-27-30
11-17-19-27
11-17-19-30
11-17-19-27-30
11-17-26-27
11-17-26-30
11-17-26-27-30
11-17-27
11-17-30
11-17-27-30
11-19-26-27
11-19-26-30
11-19-26-27-30
11-19-27
11-19-30
11-19-27-30
11-26-27
11-26-30
11-26-27-30
11-27
11-30
11-27-30
15-17-19-26-27
15-17-19-26-30
15-17-19-26-27-30
15-17-19-27
15-17-19-30
15-17-19-27-30
15-17-26-27
15-17-26-30
15-17-26-27-30
15-17-27
15-17-30
15-17-27-30
15-19-26-27
15-19-26-30
15-19-26-27-30
15-19-27
15-19-30
15-19-27-30
15-26-27
15-26-30
15-26-27-30
15-27
15-30
15-27-30
17-19-26-27
17-19-26-30
17-19-26-27-30
17-19-27
17-19-30
17-19-27-30
17-26-27
17-26-30
17-26-27-30
17-27
17-30
17-27-30
19-26-27
19-26-30
19-26-27-30
19-27
19-30
19-27-30
26-27
26-30
26-27-30
27-30

TABLE 5
All combinations of amino acid substitutions in relation to K7

Val4Ile-Leu5Val-Dha8Dhb-
Val11Ala-Tyr15Leu-Lys17Arg-
Val19Phe-Asn26His-Gly30Lys
Val4Ile-Leu5Val-Dha8Dhb-
Val11Ala-Tyr15Leu-Lys17Arg-
Val19Phe-Asn26His-Ile27Phe-
Gly30Lys
Val4Ile-Leu5Val-Dha8Dhb-
Val11Ala-Tyr15Leu-Lys17Arg-
Val19Phe-Ile27Phe
Val4Ile-Leu5Val-Dha8Dhb-
Val11Ala-Tyr15Leu-Lys17Arg-
Val19Phe-Gly30Lys
Val4Ile-Leu5Val-Dha8Dhb-
Val11Ala-Tyr15Leu-Lys17Arg-
Val19Phe-Ile27Phe-Gly30Lys
Val4Ile-Leu5Val-Dha8Dhb-
Val11Ala-Tyr15Leu-Lys17Arg-
Asn26His-Ile27Phe
Val4Ile-Leu5Val-Dha8Dhb-
Val11Ala-Tyr15Leu-Lys17Arg-
Asn26His-Gly30Lys
Val4Ile-Leu5Val-Dha8Dhb-
Val11Ala-Tyr15Leu-Lys17Arg-
Asn26His-Ile27Phe-Gly30Lys
Val4Ile-Leu5Val-Dba8Dhb-
Val11Ala-Tyr15Leu-Lys17Arg-
Ile27Phe
Val11Ala-Ile27Phe-Gly30Lys
Val4Ile-Leu5Val-Dha8Dhb-
Tyr15Leu-Lys17Arg-Val19Phe-
Asn26His-Ile27Phe
Val4Ile-Leu5Val-Dha8Dhb-
Tyr15Leu-Lys17Arg-Val19Phe-
Asn26His-Gly30Lys
Val4Ile-Leu5Val-Dha8Dhb-
Tyr15Leu-Lys17Arg-Val19Phe-
Asn26His-Ile27Phe-Gly30Lys
Val4Ile-Leu5Val-Dha8Dhb-
Tyr15Leu-Lys17Arg-Val19Phe-
Ile27Phe
Val4Ile-Leu5Val-Dha8Dhb-
Tyr15Leu-Lys17Arg-Val19Phe-
Gly30Lys
Val4Ile-Leu5Val-Dha8Dhb-
Tyr15Leu-Lys17Arg-Val19Phe-
Ile27Phe-Gly30Lys
Val4Ile-Leu5Val-Dha8Dhb-
Tyr15Leu-Lys17Arg-Asn26His-
Ile27Phe
Val4Ile-Leu5Val-Dha8Dhb-
Tyr15Leu-Lys17Arg-Asn26His-
Gly30Lys
Val4Ile-Leu5Val-Dha8Dhb-
Tyr15Leu-Lys17Arg-Asn26His-
Ile27Phe-Gly30Lys
Val4Ile-Leu5Val-Dha8Dhb-
Tyr15Leu-Lys17Arg-Ile27Phe
Val4Ile-Leu5Val-Dha8Dhb-
Tyr15Leu-Lys17Arg-Val19Phe-
Ile27Phe-Gly30Lys
Val4Ile-Leu5Val-Val11Ala-
Tyr15Leu-Lys17Arg-Asn26His-
Ile27Phe
Val4Ile-Leu5Val-Val11Ala-
Tyr15Leu-Lys17Arg-Asn26His-
Gly30Lys
Val4Ile-Leu5Val-Val11Ala-
Tyr15Leu-Lys17Arg-Asn26His-
Ile27Phe-Gly30Lys
Val4Ile-Leu5Val-Val11Ala-
Tyr15Leu-Lys17Arg-Ile27Phe
Val4Ile-Leu5Val-Val11Ala-
Tyr15Leu-Lys17Arg-Gly30Lys
Val4Ile-Leu5Val-Val11Ala-
Tyr15Leu-Lys17Arg-Ile27Phe-
Gly30Lys
Val4Ile-Leu5Val-Val11Ala-
Tyr15Leu-Val19Phe-Asn26His-
Ile27Phe
Val4Ile-Leu5Val-Val11Ala-
Tyr15Leu-Val19Phe-Asn26His-
Gly30Lys
Val4Ile-Leu5Val-Val11Ala-
Tyr15Leu-Val19Phe-Asn26His-
Ile27Phe-Gly30Lys
Val4Ile-Leu5Val-Val11Ala-
Tyr15Leu-Val19Phe-Ile27Phe
Val4Ile-Leu5Val-Val11Ala-
Tyr15Leu-Val19Phe-Gly30Lys
Val19Phe-Asn26His-Ile27Phe-
Gly30Lys
Val4Ile-Leu5Val-Tyr15Leu-
Val19Phe-Ile27Phe
Val4Ile-Leu5Val-Tyr15Leu-
Val19Phe-Gly30Lys
Val4Ile-Leu5Val-Tyr15Leu-
Val19Phe-Ile27Phe-Gly30Lys
Val4Ile-Leu5Val-Tyr15Leu-
Asn26His-Ile27Phe
Val4Ile-Leu5Val-Tyr15Leu-
Asn26His-Gly30Lys
Val4Ile-Leu5Val-Tyr15Leu-
Asn26His-Ile27Phe-Gly30Lys
Val4Ile-Leu5Val-Tyr15Leu-
Ile27Phe
Val4Ile-Leu5Val-Tyr15Leu-
Gly30Lys
Val4Ile-Leu5Val-Tyr15Leu-
Ile27Phe-Gly30Lys
Val4Ile-Leu5Val-Lys17Arg-
Val19Phe-Asn26His-Ile27Phe
Val4Ile-Leu5Val-Lys17Arg-
Val19Phe-Asn26His-Gly30Lys
Val4Ile-Leu5Val-Lys17Arg-
Val119Phe-Asn26His-Ile27Phe-
Gly30Lys
Val4Ile-Leu5Val-Lys17Arg-
Val19Phe-Ile27Phe
Val4Ile-Leu5Val-Lys17Arg-
Val19Phe-Gly30Lys
Val4Ile-Dha8Dhb-Val11Ala-
Lys17Arg-Val19Phe-Ile27Phe
Val4Ile-Dha8Dhb-Val11Ala-
Lys17Arg-Val19Phe-Gly30Lys
Val4Ile-Dha8Dhb-Val11Ala-
Lys17Arg-Val19Phe-Ile27Phe-
Gly30Lys
Val4Ile-Dha8Dhb-Val11Ala-
Lys17Arg-Asn26His-Ile27Phe
Val4Ile-Dha8Dhb-Val11Ala-
Lys17Arg-Asn26His-Gly30Lys
Val4Ile-Dha8Dhb-Val11Ala-
Lys17Arg-Asn26His-Ile27Phe-
Gly30Lys
Val4Ile-Dha8Dhb-Val11Ala-
Lys17Arg-Ile27Phe
Val4Ile-Dha8Dhb-Val11Ala-
Lys17Arg-Gly30Lys
Val4Ile-Dha8Dhb-Val11Ala-
Lys17Arg-Ile27Phe-Gly30Lys
Val4Ile-Dha8Dhb-Val11Ala-
Val19Phe-Asn26His-
Ile27PheVal4Ile-Dha8Dhb-
Val11Ala-Val19Phe-Asn26His-
Gly30Lys
Val4Ile-Dha8Dhb-Val11Ala-
Val19Phe-Asn26His-Ile27Phe-
Gly30Lys
Val4Ile-Dha8Dhb-Val11Ala-
Val19phe-Ile27Phe
Val4Ile-Dha8Dhb-Val11Ala-
Gly30Lys
Val4Ile-Dha8Dhb-Val19Phe-
Ile27Phe-Gly30Lys
Val4Ile-Dha8Dhb-Asn26His-
Ile27Phe
Val4Ile-Dha8Dhb-Asn26His-
Gly30Lys
Val4Ile-Dha8Dhb-Asn26His-
Ile27Phe-Gly30Lys
Val4Ile-Dha8Dhb-Ile27Phe
Val4Ile-Dha8Dhb-Gly30Lys
Val4Ile-Dha8Dhb-Ile27Phe-
Gly30Lys
Val4Ile-Val11Ala-Tyr15Leu-
Lys17Arg-Val19Phe-Asn26His-
Ile27Phe
Val4Ile-Val11Ala-Tyr15Leu-
Lys17Arg-Val19Phe-Asn26His-
Gly30Lys
Val4Ile-Val11Ala-Tyr15Leu-
Lys17Arg-Val19Phe-Asn26His-
Ile27Phe-Gly30Lys
Val4Ile-Val11Ala-Tyr15Leu-
Lys17Arg-Val19Phe-Ile27Phe
Val4Ile-Val11Ala-Tyr15Leu-
Lys17Arg-Val19Phe-Gly30Lys
Val4Ile-Val11Ala-Tyr15Leu-
Lys17Arg-Val19Phe-Ile27Phe-
Gly30Lys
Val4Ile-Val11Ala-Tyr15Leu-
Lys17Arg-Asn26His-Ile27Phe
Val4Ile-Tyr15Leu-Lys17Arg-
Asn26His-Ile27Phe-Gly30Lys
Val4Ile-Tyr15Leu-Lys17Arg-
Ile27Phe
Val4Ile-Tyr15Leu-Lys17Arg-
Gly30Lys
Val4Ile-Tyr15Leu-Lys17Arg-
Ile27Phe-Gly30Lys
Val4Ile-Tyr15Leu-Val19Phe-
Asn26His-Ile27Phe
Val4Ile-Tyr15Leu-Val19Phe-
Asn26His-Gly30Lys
Val4Ile-Tyr15Leu-Val19Phe-
Asn26His-Ile27Phe-Gly30Lys
Val4Ile-Tyr15Leu-Val19Phe-
Ile27Phe
Val4Ile-Tyr15Leu-Val19Phe-
Gly30Lys
Val4Ile-Tyr15Leu-Val19Phe-
Ile27Phe-Gly30Lys
Val4Ile-Tyr15Leu-Asn26His-
Ile27Phe
Val4Ile-Tyr15Leu-Asn26His-
Gly30Lys
Val4Ile-Tyr15Leu-Asn26His-
Ile27Phe-Gly30Lys
Val4Ile-Tyr15Leu-Ile27Phe
Val4Ile-Tyr15Leu-Gly30Lys
Val4Ile-Tyr15Leu-Ile27Phe-
Gly30Lys
Val4Ile-Lys17Arg-Val19Phe-
Leu5Val-Dha8Dhb-Val11Ala-
Lys17Arg-Val19Phe-Ile27Phe
Leu5Val-Dha8Dhb-Val11Ala-
Lys17Arg-Val19Phe-Gly30Lys
Leu5Val-Dha8Dhb-Val11Ala-
Lys17Arg-Val19Phe-Ile27Phe-
Gly30Lys
Leu5Val-Dha8Dhb-Val11Ala-
Lys17Arg-Asn26His-Ile27Phe
Leu5Val-Dha8Dhb-Val11Ala-
Lys17Arg-Asn26His-Gly30Lys
Leu5Val-Dha8Dhb-Val11Ala-
Lys17Arg-Asn26His-Ile27Phe-
Gly30Lys
Leu5Val-Dha8Dhb-Val11Ala-
Lys17Arg-Ile27Phe
Leu5Val-Dha8Dhb-Val11Ala-
Lys17Arg-Gly30Lys
Leu5Val-Dha8Dhb-Val11Ala-
Lys17Arg-Ile27Phe-Gly30Lys
Leu5Val-Dha8Dhb-Val11Ala-
Val19Phe-Asn26His-Ile27Phe
Leu5Val-Dha8Dhb-Val11Ala-
Val19Phe-Asn26His-Gly30Lys
Leu5Val-Dha8Dhb-Val11Ala-
Val19Phe-Asn26His-Ile27Phe-
Gly30Lys
Leu5Val-Dha8Dhb-Val11Ala-
Val19Phe-Ile27Phe
Leu5Val-Dha8Dhb-Val11Ala-
Val19Phe-Gly30Lys
Leu5Val-Dha8Dhb-Val19Phe-
Ile27Phe-Gly30Lys
Leu5Val-Dha8Dhb-Asn26His-
Ile27Phe
Leu5Val-Dha8Dhb-Asn26His-
Gly30Lys
Leu5Val-Dha8Dhb-Asn26His-
Ile27Phe-Gly30Lys
Leu5Val-Dha8Dhb-Ile27Phe
Leu5Val-Dha8Dhb-Gly30Lys
Leu5Val-Dha8Dhb-Ile27Phe-
Gly30Lys
Leu5Val-Val11Ala-Tyr15Leu-
Lys17Arg-Val19Phe-Asn26His-
Ile27Phe
Leu5Val-Val11Ala-Tyr15Leu-
Lys17Arg-Val19Phe-Asn26His-
Gly30Lys
Leu5Val-Val11Ala-Tyr15Leu-
Lys17Arg-Val19Phe-Asn26His-
Ile27Phe-Gly30Lys
Leu5Val-Val11Ala-Tyr15Leu-
Lys17Arg-Val19Phe-Ile27Phe
Leu5Val-Val11Ala-Tyr15Leu-
Lys17Arg-Val19Phe-Gly30Lys
Leu5Val-Val11Ala-Tyr15Leu-
Lys17Arg-Val19Phe-Ile27Phe-
Gly30Lys
Leu5Val-Val11Ala-Tyr15Leu-
Lys17Arg-Asn26His-Ile27Phe
Leu5Val-Tyr15Leu-Lys17Arg-
Asn26His-Gly30Lys
Leu5Val-Tyr15Leu-Lys17Arg-
Asn26His-Ile27Phe-Gly30Lys
Leu5Val-Tyr15Leu-Lys17Arg-
Ile27Phe
Leu5Val-Tyr15Leu-Lys17Arg-
Gly30Lys
Leu5Val-Tyr15Leu-Lys17Arg-
Ile27Phe-Gly30Lys
Leu5Val-Tyr15Leu-Val19Phe-
Asn26His-Ile27Phe
Leu5Val-Tyr15Leu-Val19Phe-
Asn26His-Gly30Lys
Leu5Val-Tyr15Leu-Val19Phe-
Asn26His-Ile27Phe-Gly30Lys
Leu5Val-Tyr15Leu-Val19Phe-
Ile27Phe
Leu5Val-Tyr15Leu-Val19Phe-
Gly30Lys
Leu5Val-Tyr15Leu-Val19Phe-
Ile27Phe-Gly30Lys
Leu5Val-Tyr15Leu-Asn26His-
Ile27Phe
Leu5Val-Tyr15Leu-Asn26His-
Gly30Lys
Leu5Val-Tyr15Leu-Asn26His-
Ile27Phe-Gly30Lys
Leu5Val-Tyr15Leu-Ile27Phe
Leu5Val-Tyr15Leu-Gly30Lys
Leu5Val-Tyr15Leu-Ile27Phe-
Val19Phe-Ile27Phe
Dha8Dhb-Val11Ala-Lys17Arg-
Val19Phe-Gly30Lys
Dha8Dhb-Val11Ala-Lys17Arg-
Val19Phe-Ile27Phe-Gly30Lys
Dha8Dhb-Val11Ala-Lys17Arg-
Asn26His-Ile27Phe
Dha8Dhb-Val11Ala-Lys17Arg-
Asn26His-Gly30Lys
Dha8Dhb-Val11Ala-Lys17Arg-
Asn26His-Ile27Phe-Gly30Lys
Dha8Dhb-Val11Ala-Lys17Arg-
Ile27Phe
Dha8Dhb-Val11Ala-Lys17Arg-
Gly30Lys
Dha8Dhb-Val11Ala-Lys17Arg-
Ile27Phe-Gly30Lys
Dha8Dhb-Val11Ala-Val19Phe-
Asn26His-Ile27Phe
Dha8Dhb-Val11Ala-Val19Phe-
Asn26His-Gly30Lys
Dha8Dhb-Val11Ala-Val19Phe-
Asn26His-Ile27Phe-Gly30Lys
Dha8Dhb-Val11Ala-Val19Phe-
Ile27Phe
Dha8Dhb-Val11Ala-Val19Phe-
Gly30Lys
Dha8Dhb-Val11Ala-Val19Phe-
Ile27Phe-Gly30Lys
Dha8Dhb-Val11Ala-Asn26His-
Ile27Phe
Dha8Dhb-Val11Ala-Asn26His-
Val11Ala-Tyr15Leu-Lys17Arg-
Ile27Phe
Val11Ala-Tyr15Leu-Lys17Arg-
Gly30Lys
Val11Ala-Tyr15Leu-Lys17Arg-
Ile27Phe-Gly30Lys
Val11Ala-Tyr15Leu-Val19Phe-
Asn26His-Ile27Phe
Val11Ala-Tyr15Leu-Val19Phe-
Asn26His-Gly30Lys
Val11Ala-Tyr15Leu-Val19Phe-
Asn26His-Ile27Phe-Gly30Lys
Val11Ala-Tyr15Leu-Val19Phe-
Ile27Phe
Val11Ala-Tyr15Leu-Val19Phe-
Gly30Lys
Val11Ala-Tyr15Leu-Val19Phe-
Ile27Phe-Gly30Lys
Val11Ala-Tyr15Leu-Asn26His-
Ile27Phe
Val11Ala-Tyr15Leu-Asn26His-
Gly30Lys
Val11Ala-Tyr15Leu-Asn26His-
Ile27Phe-Gly30Lys
Val11Ala-Tyr15Leu-Ile27Phe
Val11Ala-Tyr15Leu-Gly30Lys
Val11Ala-Tyr15Leu-Ile27Phe-
Gly30Lys
Val11Ala-Lys17Arg-Val19Phe-
Asn26His-Ile27Phe
Val11Ala-Lys17Arg-Val19Phe-
Asn26His-Gly30Lys
Val4Ile-Leu5Val-Dha8Dhb-Val11Ala-
Tyr15Leu-Lys17Arg-Gly30Lys
Val4Ile-Leu5Val-Dha8Dhb-Val11Ala-
Tyr15Leu-Lys17Arg-Ile27Phe-Gly30Lys
Val4Ile-Leu5Val-Dha8Dhb-Val11Ala-
Tyr15Leu-Val19Phe-Asn26His-Ile27Phe
Val4Ile-Leu5Val-Dha8Dhb-Val11Ala-
Tyr15Leu-Val19Phe-Asn26His-Gly30Lys
Val4Ile-Leu5Val-Dha8Dhb-Val11Ala-
Tyr15Leu-Val19Phe-Asn26His-Ile27Phe-
Gly30Lys
Val4Ile-Leu5Val-Dha8Dhb-Val11Ala-
Tyr15Leu-Val19Phe-Ile27Phe
Val4Ile-Leu5Val-Dha8Dhb-Val11Ala-
Tyr15Leu-Val19Phe-Gly30Lys
Val4Ile-Leu5Val-Dha8Dhb-Val11Ala-
Tyr15Leu-Val19Phe-Ile27Phe-Gly30Lys
Val4Ile-Leu5Val-Dha8Dhb-Val11Ala-
Tyr15Leu-Asn26His-Ile27Phe
Val4Ile-Leu5Val-Dha8Dhb-Val11Ala-
Tyr15Leu-Asn26His-Gly30Lys
Val4Ile-Leu5Val-Dha8Dhb-Val11Ala-
Tyr15Leu-Asn26His-Ile27Phe-Gly30Lys
Val4Ile-Leu5Val-Dha8Dhb-Val11Ala-
Tyr15Leu-Ile27PheVal4Ile-Leu5Val-
Dha8Dhb-Val11Ala-Tyr15Leu-Gly30Lys
Val4Ile-Leu5Val-Dha8Dhb-Val11Ala-
Tyr15Leu-Ile27Phe-Gly30LysVal4Ile-
Tyr15Leu-Lys17Arg-Gly30Lys
Val4Ile-Leu5Val-Dha8Dhb-Tyr15Leu-
Lys17Arg-Ile27Phe-Gly30Lys
Val4Ile-Leu5Val-Dha8Dhb-Tyr15Leu-
Val19Phe-Asn26His-Ile27Phe
Val4Ile-Leu5Val-Dha8Dhb-Tyr15Leu-
Val19Phe-Asn26His-Gly30Lys
Val4Ile-Leu5Val-Dha8Dhb-Tyr15Leu-
Val19Phe-Asn26His-Ile27Phe-Gly30Lys
Val4Ile-Leu5Val-Dha8Dhb-Tyr15Leu-
Val19Phe-Ile27Phe
Val4Ile-Leu5Val-Dha8Dhb-Tyr15Leu-
Val19Phe-Gly30Lys
Val4Ile-Leu5Val-Dha8Dhb-Tyr15Leu-
Val19Phe-Ile27Phe-Gly30LysVal4Ile-
Leu5Val-Dha8Dhb-Tyr15Leu-Asn26His-
Ile27Phe
Val4Ile-Leu5Val-Dha8Dhb-Tyr15Leu-
Asn26His-Gly30Lys
Val4Ile-Leu5Val-Dha8Dhb-Tyr15Leu-
Asn26His-Ile27Phe-Gly30Lys
Val4Ile-Leu5Val-Dha8Dhb-Tyr15Leu-
Ile27Phe
Val4Ile-Leu5Val-Dha8Dhb-Tyr15Leu-
Gly30Lys
Val4Ile-Leu5Val-Dha8Dhb-Tyr15Leu-
Ile27Phe-Gly30Lys
Val4Ile-Leu5Val-Dha8Dhb-Lys17Arg-
Val19Phe-Asn26His-Ile27Phe
Val4Ile-Leu5Val-Dha8Dhb-Lys17Arg-
Val19Phe-Asn26His-Gly30Lys
Val4Ile-Leu5Val-Val11Ala-Tyr15Leu-
Val19Phe-Ile27Phe-Gly30Lys
Val4Ile-Leu5Val-Val11Ala-Tyr15Leu-
Asn26His-Ile27Phe
Val4Ile-Leu5Val-Val11Ala-Tyr15Leu-
Asn26His-Gly30Lys
Val4Ile-Leu5Val-Val11Ala-Tyr15Leu-
Asn26His-Ile27Phe-Gly30Lys
Val4Ile-Leu5Val-Val11Ala-Tyr15Leu-
Ile27Phe
Val4Ile-Leu5Val-Val11Ala-Tyr15Leu-
Gly30Lys
Val4Ile-Leu5Val-Val11Ala-Tyr15Leu-
Ile27Phe-Gly30Lys
Val4Ile-Leu5Val-Val11Ala-Lys17Arg-
Val19Phe-Asn26His-Ile27Phe
Val4Ile-Leu5Val-Val11Ala-Lys17Arg-
Val19Phe-Asn26His-Gly30Lys
Val4Ile-Leu5Val-Val11Ala-Lys17Arg-
Val19Phe-Asn26His-Ile27Phe-Gly30Lys
Val4Ile-Leu5Val-Val11Ala-Lys17Arg-
Val19Phe-Ile27Phe
Val4Ile-Leu5Val-Val11Ala-Lys17Arg-
Val19Phe-Gly30Lys
Val4Ile-Leu5Val-Val11Ala-Lys17Arg-
Val19Phe-Ile27Phe-Gly30Lys
Val4Ile-Leu5Val-Val11Ala-Lys17Arg-
Asn26His-Ile27Phe
Val4Ile-Leu5Val-Val11Ala-Lys17Arg-
Asn26His-Gly30Lys
Val4Ile-Leu5Val-Val11Ala-Lys17Arg-
Val4Ile-Leu5Val-Lys17Arg-Val19Phe-
Ile27Phe-Gly30Lys
Val4Ile-Leu5Val-Lys17Arg-Asn26His-
Ile27Phe
Val4Ile-Leu5Val-Lys17Arg-Asn26His-
Gly30Lys
Val4Ile-Leu5Val-Lys17Arg-Asn26His-
Ile27Phe-Gly30Lys
Val4Ile-Leu5Val-Lys17Arg-Ile27Phe
Val4Ile-Leu5Val-Lys17Arg-Gly30Lys
Val4Ile-Leu5Val-Lys17Arg-Ile27Phe-
Gly30Lys
Val4Ile-Leu5Val-Val19Phe-Asn26His-
Ile27Phe
Val4Ile-Leu5Val-Val19Phe-Asn26His-
Gly30Lys
Val4Ile-Leu5Val-Val19Phe-Asn26His-
Ile27Phe-Gly30Lys
Val4Ile-Leu5Val-Val19Phe-Ile27Phe
Val4Ile-Leu5Val-Val19Phe-Gly30Lys
Val4Ile-Leu5Val-Val19Phe-Ile27Phe-
Gly30Lys
Val4Ile-Leu5Val-Asn26His-Ile27Phe
Val4Ile-Leu5Val-Asn26His-Gly30Lys
Val4Ile-Leu5Val-Asn26His-Ile27Phe-
Gly30Lys
Val4Ile-Leu5Val-Ile27Phe
Val4Ile-Leu5Val-Gly30Lys
Val4Ile-Leu5Val-Ile27Phe-Gly30Lys
Val4Ile-Dha8Dhb-Val11Ala-Tyr15Leu-
Lys17Arg-Val19Phe-Asn26His-Ile27Phe
Val19Phe-Gly30Lys
Val4Ile-Dha8Dhb-Val11Ala-Val19Phe-
Ile27Phe-Gly30Lys
Val4Ile-Dha8Dhb-Val11Ala-Asn26His-
Ile27Phe
Val4Ile-Dha8Dhb-Val11Ala-Asn26His-
Gly30Lys
Val4Ile-Dha8Dhb-Val11Ala-Asn26His-
Ile27Phe-Gly30Lys
Val4Ile-Dha8Dhb-Val11Ala-Ile27Phe
Val4Ile-Dha8Dhb-Val11Ala-Gly30Lys
Val4Ile-Dha8Dhb-Val11Ala-Ile27Phe-
Gly30Lys
Val4Ile-Dha8Dhb-Tyr15Leu-Lys17Arg-
Val19Phe-Asn26His-Ile27Phe
Val4Ile-Dha8Dhb-Tyr15Leu-Lys17Arg-
Val19Phe-Asn26His-Gly30Lys
Val4Ile-Dha8Dhb-Tyr15Leu-Lys17Arg-
Val19Phe-Asn26His-Ile27Phe-Gly30Lys
Val4Ile-Dha8Dhb-Tyr15Leu-Lys17Arg-
Val19Phe-Ile27Phe
Val4Ile-Dha8Dhb-Tyr15Leu-Lys17Arg-
Val19Phe-Gly30Lys
Val4Ile-Dha8Dhb-Tyr15Leu-Lys17Arg-
Val19Phe-Ile27Phe-Gly30Lys
Val4Ile-Dha8Dhb-Tyr15Leu-Lys17Arg-
Asn26His-Ile27Phe
Val4Ile-Dha8Dhb-Tyr15Leu-Lys17Arg-
Asn26His-Gly30Lys
Val4Ile-Dha8Dhb-Tyr15Leu-Lys17Arg-
Asn26His-Ile27Phe-Gly30Lys
Val4Ile-Val11Ala-Tyr15Leu-Lys17Arg-
Asn26His-Gly30Lys
Val4Ile-Val11Ala-Tyr15Leu-Lys17Arg-
Asn26His-Ile27Phe-Gly30Lys
Val4Ile-Val11Ala-Tyr15Leu-Lys17Arg-
Ile27Phe
Val4Ile-Val11Ala-Tyr15Leu-Lys17Arg-
Gly30Lys
Val4Ile-Val11Ala-Tyr15Leu-Lys17Arg-
Ile27Phe-Gly30Lys
Val4Ile-Val11Ala-Tyr15Leu-Val19Phe-
Asn26His-Ile27Phe
Val4Ile-Val11Ala-Tyr15Leu-Val19Phe-
Asn26His-Gly30Lys
Val4Ile-Val11Ala-Tyr15Leu-Val19Phe-
Asn26His-Ile27Phe-Gly30Lys
Val4Ile-Val11Ala-Tyr15Leu-Val19Phe-
Ile27Phe
Val4Ile-Val11Ala-Tyr15Leu-Val19Phe-
Gly30Lys
Val4Ile-Val11Ala-Tyr15Leu-Val19Phe-
Ile27Phe-Gly30Lys
Val4Ile-Val11Ala-Tyr15Leu-Asn26His-
Ile27Phe
Val4Ile-Val11Ala-Tyr15Leu-Asn26His-
Gly30Lys
Val4Ile-Val11Ala-Tyr15Leu-Asn26His-
Ile27Phe-Gly30Lys
Val4Ile-Val11Ala-Tyr15Leu-Ile27Phe
Val4Ile-Val11Ala-Tyr15Leu-Gly30Lys
Val4Ile-Val11Ala-Tyr15Leu-Ile27Phe-
Asn26His-Ile27Phe
Val4Ile-Lys17Arg-Val19Phe-Asn26His-
Gly30Lys
Val4Ile-Lys17Arg-Val19Phe-Asn26His-
Ile27Phe-Gly30Lys
Val4Ile-Lys17Arg-Val19Phe-Ile27Phe
Val4Ile-Lys17Arg-Val19Phe-Gly30Lys
Val4Ile-Lys17Arg-Val19Phe-Ile27Phe-
Gly30Lys
Val4Ile-Lys17Arg-Asn26His-Ile27Phe
Val4Ile-Lys17Arg-Asn26His-Gly30Lys
Val4Ile-Lys17Arg-Asn26His-Ile27Phe-
Gly30Lys
Val4Ile-Lys17Arg-Ile27Phe
Val4Ile-Lys17Arg-Gly30Lys
Val4Ile-Lys17Arg-Ile27Phe-Gly30Lys
Val4Ile-Val19Phe-Asn26His-Ile27Phe
Val4Ile-Val19Phe-Asn26His-Gly30Lys
Val4Ile-Val19Phe-Asn26His-Ile27Phe-
Gly30Lys
Val4Ile-Val19Phe-Ile27Phe
Val4Ile-Val19Phe-Gly30Lys
Val4Ile-Val19Phe-Ile27Phe-Gly30Lys
Val4Ile-Asn26His-Ile27Phe
Val4Ile-Asn26His-Gly30Lys
Val4Ile-Asn26His-Ile27Phe-Gly30Lys
Val4Ile-Ile27Phe
Val4Ile-Gly30Lys
Val4Ile-Ile27Phe-Gly30Lys
Leu5Val-Dha8Dhb-Val11Ala-Tyr15Leu-
Lys17Arg-Val19Phe-Asn26His-Ile27Phe
Leu5Val-Dha8Dhb-Val11Ala-Val19Phe-
Ile27Phe-Gly30Lys
Leu5Val-Dha8Dhb-Val11Ala-Asn26His-
Ile27Phe
Leu5Val-Dha8Dhb-Val11Ala-Asn26His-
Gly30Lys
Leu5Val-Dha8Dhb-Val11Ala-Asn26His-
Ile27Phe-Gly30Lys
Leu5Val-Dha8Dhb-Val11Ala-Ile27Phe
Leu5Val-Dha8Dhb-Val11Ala-Gly30Lys
Leu5Val-Dha8Dhb-Val11Ala-Ile27Phe-
Gly30Lys
Leu5Val-Dha8Dhb-Tyr15Leu-Lys17Arg-
Val19Phe-Asn26His-Ile27Phe
Leu5Val-Dha8Dhb-Tyr15Leu-Lys17Arg-
Val19Phe-Asn26His-Gly30Lys
Leu5Val-Dha8Dhb-Tyr15Leu-Lys17Arg-
Val19Phe-Asn26His-Ile27Phe-Gly30Lys
Leu5Val-Dha8Dhb-Tyr15Leu-Lys17Arg-
Val19Phe-Ile27Phe
Leu5Val-Dha8Dhb-Tyr15Leu-Lys17Arg-
Val19Phe-Gly30Lys
Leu5Val-Dha8Dhb-Tyr15Leu-Lys17Arg-
Val19Phe-Ile27Phe-Gly30Lys
Leu5Val-Dha8Dhb-Tyr15Leu-Lys17Arg-
Asn26His-Ile27Phe
Leu5Val-Dha8Dhb-Tyr15Leu-Lys17Arg-
Asn26His-Gly30Lys
Leu5Val-Dha8Dhb-Tyr15Leu-Lys17Arg-
Asn26His-Ile27Phe-Gly30Lys
Leu5Val-Dha8Dhb-Tyr15Leu-Lys17Arg-
Leu5Val-Val11Ala-Tyr15Leu-Lys17Arg-
Asn26His-Gly30Lys
Leu5Val-Val11Ala-Tyr15Leu-Lys17Arg-
Asn26His-Ile27Phe-Gly30Lys
Leu5Val-Val11Ala-Tyr15Leu-Lys17Arg-
Ile27Phe
Leu5Val-Val11Ala-Tyr15Leu-Lys17Arg-
Gly30Lys
Leu5Val-Val11Ala-Tyr15Leu-Lys17Arg-
Ile27Phe-Gly30Lys
Leu5Val-Val11Ala-Tyr15Leu-Val19Phe-
Asn26His-Ile27Phe
Leu5Val-Val11Ala-Tyr15Leu-Val19Phe-
Asn26His-Gly30Lys
Leu5Val-Val11Ala-Tyr15Leu-Val19Phe-
Asn26His-Ile27Phe-Gly30Lys
Leu5Val-Val11Ala-Tyr15Leu-Val19Phe-
Ile27Phe
Leu5Val-Val11Ala-Tyr15Leu-Val19Phe-
Gly30Lys
Leu5Val-Val11Ala-Tyr15Leu-Val19Phe-
Ile27Phe-Gly30Lys
Leu5Val-Val11Ala-Tyr15Leu-Asn26His-
Ile27Phe
Leu5Val-Val11Ala-Tyr15Leu-Asn26His-
Gly30Lys
Leu5Val-Val11Ala-Tyr15Leu-Asn26His-
Ile27Phe-Gly30Lys
Leu5Val-Val11Ala-Tyr15Leu-Ile27Phe
Leu5Val-Val11Ala-Tyr15Leu-Gly30Lys
Gly30Lys
Leu5Val-Lys17Arg-Val19Phe-Asn26His-
Ile27Phe
Leu5Val-Lys17Arg-Val19Phe-Asn26His-
Gly30Lys
Leu5Val-Lys17Arg-Val19Phe-Asn26His-
Ile27Phe-Gly30Lys
Leu5Val-Lys17Arg-Val19Phe-Ile27Phe
Leu5Val-Lys17Arg-Val19Phe-Gly30Lys
Leu5Val-Lys17Arg-Val19Phe-Ile27Phe-
Gly30Lys
Leu5Val-Lys17Arg-Asn26His-Ile27Phe
Leu5Val-Lys17Arg-Asn26His-Gly30Lys
Leu5Val-Lys17Arg-Asn26His-Ile27Phe-
Gly30Lys
Leu5Val-Lys17Arg-Ile27Phe
Leu5Val-Lys17Arg-Gly30Lys
Leu5Val-Lys17Arg-Ile27Phe-Gly30Lys
Leu5Val-Val19Phe-Asn26His-Ile27Phe
Leu5Val-Val19Phe-Asn26His-Gly30Lys
Leu5Val-Val19Phe-Asn26His-Ile27Phe-
Gly30Lys
Leu5Val-Val19Phe-Ile27Phe
Leu5Val-Val19Phe-Gly30Lys
Leu5Val-Val19Phe-Ile27Phe-Gly30Lys
Leu5Val-Asn26His-Ile27Phe
Leu5Val-Asn26His-Gly30Lys
Leu5Val-Asn26His-Ile27Phe-Gly30Lys
Leu5Val-Ile27Phe
Leu5Val-Gly30Lys
Leu5Val-Ile27Phe-Gly30Lys
Gly30Lys
Dha8Dhb-Val11Ala-Asn26His-Ile27Phe-
Gly30Lys
Dha8Dhb-Val11Ala-Ile27Phe
Dha8Dhb-Val11Ala-Gly30Lys
Dha8Dhb-Val11Ala-Ile27Phe-Gly30Lys
Dha8Dhb-Tyr15Leu-Lys17Arg-Val19Phe-
Asn26His-Ile27Phe
Dha8Dhb-Tyr15Leu-Lys17Arg-Val19Phe-
Asn26His-Gly30Lys
Dha8Dhb-Tyr15Leu-Lys17Arg-Val19Phe-
Asn26His-Ile27Phe-Gly30Lys
Dha8Dhb-Tyr15Leu-Lys17Arg-Val19Phe-
Ile27Phe
Dha8Dhb-Tyr15Leu-Lys17Arg-Val19Phe-
Gly30Lys
Dha8Dhb-Tyr15Leu-Lys17Arg-Val19Phe-
Ile27Phe-Gly30Lys
Dha8Dhb-Tyr15Leu-Lys17Arg-Asn26His-
Ile27Phe
Dha8Dhb-Tyr15Leu-Lys17Arg-Asn26His-
Gly30Lys
Dha8Dhb-Tyr15Leu-Lys17Arg-Asn26His-
Ile27Phe-Gly30Lys
Dha8Dhb-Tyr15Leu-Lys17Arg-Ile27Phe
Dha8Dhb-Tyr15Leu-Lys17Arg-Gly30Lys
Dha8Dhb-Tyr15Leu-Lys17Arg-Ile27Phe-
Gly30Lys
Dha8Dhb-Tyr15Leu-Val19Phe-Asn26His-
Ile27Phe
Dha8Dhb-Tyr15Leu-Val19Phe-Asn26His-
Gly30Lys
Val11Ala-Lys17Arg-Val19Phe-Asn26His-
Ile27Phe-Gly30Lys
Val11Ala-Lys17Arg-Val19Phe-Ile27Phe
Val11Ala-Lys17Arg-Val19Phe-Gly30Lys
Val11Ala-Lys17Arg-Val19Phe-Ile27Phe-
Gly30Lys
Val11Ala-Lys17Arg-Asn26His-Ile27Phe
Val11Ala-Lys17Arg-Asn26His-Gly30Lys
Val11Ala-Lys17Arg-Asn26His-Ile27Phe-
Gly30Lys
Val11Ala-Lys17Arg-Ile27Phe
Val11Ala-Lys17Arg-Gly30Lys
Val11Ala-Lys17Arg-Ile27Phe-Gly30Lys
Val11Ala-Val19Phe-Asn26His-Ile27Phe
Val11Ala-Val19Phe-Asn26His-Gly30Lys
Val11Ala-Val19Phe-Asn26His-Ile27Phe-
Gly30Lys
Val11Ala-Val19Phe-Ile27Phe
Val11Ala-Val19Phe-Gly30Lys
Val11Ala-Val19Phe-Ile27Phe-Gly30Lys
Val11Ala-Asn26His-Ile27Phe
Val11Ala-Asn26His-Gly30Lys
Val11Ala-Asn26His-Ile27Phe-Gly30Lys
Val11Ala-Ile27Phe
Val11Ala-Gly30Lys
Val11Ala-Ile27Phe-Gly30Lys
Tyr15Leu-Lys17Arg-Val19Phe-Asn26His-
Ile27Phe
Tyr15Leu-Lys17Arg-Val19Phe-Asn26His-
Gly30Lys
Tyr15Leu-Lys17Arg-Val19Phe-Asn26His-
Leu5Val-Dha8Dhb-Val11Ala-Lys17Arg-
Val19Phe-Asn26His-Ile27Phe
Val4Ile-Leu5Val-Dha8Dhb-Val11Ala-
Lys17Arg-Val19Phe-Asn26His-
Gly30Lys
Val4Ile-Leu5Val-Dha8Dhb-Val11Ala-
Lys17Arg-Val19Phe-Asn26His-
Ile27Phe-Gly30Lys
Val4Ile-Leu5Val-Dha8Dhb-Val11Ala-
Lys17Arg-Val19Phe-Ile27Phe
Val4Ile-Leu5Val-Dha8Dhb-Val11Ala-
Lys17Arg-Val19Phe-Gly30Lys
Val4Ile-Leu5Val-Dha8Dhb-Val11Ala-
Lys17Arg-Val19Phe-Ile27Phe-
Gly30Lys
Val4Ile-Leu5Val-Dha8Dhb-Val11Ala-
Lys17Arg-Asn26His-Ile27PheVal4Ile-
Leu5Val-Dha8Dhb-Val11Ala-Lys17Arg-
Asn26His-Gly30Lys
Val4Ile-Leu5Val-Dha8Dhb-Val11Ala-
Lys17Arg-Asn26His-Ile27Phe-
Gly30Lys
Val4Ile-Leu5Val-Dha8Dhb-Val11Ala-
Lys17Arg-Ile27Phe
Val4Ile-Leu5Val-Dha8Dhb-Val11Ala-
Lys17Arg-Gly30Lys
Val4Ile-Leu5Val-Dha8Dhb-Val11Ala-
Lys17Arg-Ile27Phe-Gly30Lys
Val4Ile-Leu5Val-Dha8Dhb-Lys17Arg-
Val19Phe-Asn26His-Ile27Phe-
Gly30Lys
Val4Ile-Leu5Val-Dha8Dhb-Lys17Arg-
Val19Phe-Ile27Phe
Val4Ile-Leu5Val-Dha8Dhb-Lys17Arg-
Val19Phe-Gly30Lys
Val4Ile-Leu5Val-Dha8Dhb-Lys17Arg-
Val19Phe-Ile27Phe-Gly30Lys
Val4Ile-Leu5Val-Dha8Dhb-Lys17Arg-
Asn26His-Ile27Phe
Val4Ile-Leu5Val-Dha8Dhb-Lys17Arg-
Asn26His-Gly30Lys
Val4Ile-Leu5Val-Dha8Dhb-Lys17Arg-
Asn26His-Ile27Phe-Gly30Lys
Val4Ile-Leu5Val-Dha8Dhb-Lys17Arg-
Ile27Phe
Val4Ile-Leu5Val-Dha8Dhb-Lys17Arg-
Gly30Lys
Val4Ile-Leu5Val-Dha8Dhb-Lys17Arg-
Ile27Phe-Gly30Lys
Val4Ile-Leu5Val-Dha8Dhb-Val19Phe-
Asn26His-Ile27Phe
Val4Ile-Leu5Val-Dha8Dhb-Val19Phe-
Asn26His-Gly30Lys
Val4Ile-Leu5Val-Dha8Dhb-Val19Phe-
Asn26His-Ile27Phe-Gly30Lys
Val4Ile-Leu5Val-Dha8Dhb-Val19Phe-
Ile27Phe
Val4Ile-Leu5Val-Dha8Dhb-Val19Phe-
Gly30Lys
Asn26His-Ile27Phe-Gly30Lys
Val4Ile-Leu5Val-Val11Ala-Lys17Arg-
Ile27Phe
Val4Ile-Leu5Val-Val11Ala-Lys17Arg-
Gly30Lys
Val4Ile-Leu5Val-Val11Ala-Lys17Arg-
Ile27Phe-Gly30Lys
Val4Ile-Leu5Val-Val11Ala-Val19Phe-
Asn26His-Ile27Phe
Val4Ile-Leu5Val-Val11Ala-Val19Phe-
Asn26His-Gly30Lys
Val4Ile-Leu5Val-Val11Ala-Val19Phe-
Asn26His-Ile27Phe-Gly30Lys
Val4Ile-Leu5Val-Val11Ala-Val19Phe-
Ile27Phe
Val4Ile-Leu5Val-Val11Ala-Val19Phe-
Gly30Lys
Val4Ile-Leu5Val-Val11Ala-Val19Phe-
Ile27Phe-Gly30Lys
Val4Ile-Leu5Val-Val11Ala-Asn26His-
Ile27Phe
Val4Ile-Leu5Val-Val11Ala-Asn26His-
Gly30Lys
Val4Ile-Leu5Val-Val11Ala-Asn26His-
Ile27Phe-Gly30Lys
Val4Ile-Leu5Val-Val11Ala-Ile27Phe
Val4Ile-Leu5Val-Val11Ala-Gly30Lys
Val4Ile-Leu5Val-Val11Ala-Ile27Phe-
Gly30Lys
Val4Ile-Leu5Val-Tyr15Leu-Lys17Arg-
Val19Phe-Asn26His-Ile27Phe
Val4Ile-Dha8Dhb-Val11Ala-Tyr15Leu-
Lys17Arg-Val19Phe-Asn26His-
Gly30Lys
Val4Ile-Dha8Dhb-Val11Ala-Tyr15Leu-
Lys17Arg-Val19Phe-Asn26His-
Ile27Phe-Gly30Lys
Val4Ile-Dha8Dhb-Val11Ala-Tyr15Leu-
Lys17Arg-Val19Phe-Ile27Phe
Val4Ile-Dha8Dhb-Val11Ala-Tyr15Leu-
Lys17Arg-Val19Phe-Gly30Lys
Val4Ile-Dha8Dhb-Val11Ala-Tyr15Leu-
Lys17Arg-Val19Phe-Ile27Phe-
Gly30Lys
Val4Ile-Dha8Dhb-Val11Ala-Tyr15Leu-
Lys17Arg-Asn26His-Ile27Phe
Val4Ile-Dha8Dhb-Val11Ala-Tyr15Leu-
Lys17Arg-Asn26His-Gly30Lys
Val4Ile-Dha8Dhb-Val11Ala-Tyr15Leu-
Lys17Arg-Asn26His-Ile27Phe-
Gly30Lys
Val4Ile-Dha8Dhb-Val11Ala-Tyr15Leu-
Lys17Arg-Ile27Phe
Val4Ile-Dha8Dhb-Val11Ala-Tyr15Leu-
Lys17Arg-Gly30Lys
Val4Ile-Dha8Dhb-Val11Ala-Tyr15Leu-
Lys17Arg-Ile27Phe-Gly30Lys
Val4Ile-Dha8Dhb-Val11Ala-Tyr15Leu-
Val19Phe-Asn26His-Ile27Phe
Val4Ile-Dha8Dhb-Val11Ala-Tyr15Leu-
Val19Phe-Asn26His-Gly30Lys
Val4Ile-Dha8Dhb-Val11Ala-Tyr15Leu-
Val4Ile-Dha8Dhb-Tyr15Leu-Lys17Arg-
Ile27Phe
Val4Ile-Dha8Dhb-Tyr15Leu-Lys17Arg-
Gly30Lys
Val4Ile-Dha8Dhb-Tyr15Leu-Lys17Arg-
Ile27Phe-Gly30Lys
Val4Ile-Dha8Dhb-Tyr15Leu-Val19Phe-
Asn26His-Ile27Phe
Val4Ile-Dha8Dhb-Tyr15Leu-Val19Phe-
Asn26His-Gly30Lys
Val4Ile-Dha8Dhb-Tyr15Leu-Val19Phe-
Asn26His-Ile27Phe-Gly30Lys
Val4Ile-Dha8Dhb-Tyr15Leu-Val19Phe-
Ile27Phe
Val4Ile-Dha8Dh-Tyr15Leu-Val19Phe-
Gly30Lys
Val4Ile-Dha8Dhb-Tyr15Leu-Val19Phe-
Ile27Phe-Gly30Lys
Val4Ile-Dha8Dhb-Tyr15Leu-Asn26His-
Ile27Phe
Val4Ile-Dha8Dhb-Tyr15Leu-Asn26His-
Gly30Lys
Val4Ile-Dha8Dhb-Tyr15Leu-Asn26His-
Ile27Phe-Gly30Lys
Val4Ile-Dha8Dhb-Tyr15Leu-Ile27Phe
Val4Ile-Dha8Dhb-Tyr15Leu-Gly30Lys
Val4Ile-Dha8Dhb-Tyr15Leu-Ile27Phe-
Gly30Lys
Val4Ile-Dha8Dhb-Lys17Arg-Val19Phe-
Asn26His-Ile27Phe
Val4Ile-Dha8Dhb-Lys17Arg-Val19Phe-
Gly30Lys
Val4Ile-Val11Ala-Lys17Arg-
Val19Phe-Asn26His-Ile27Phe
Val4Ile-Val11Ala-Lys17Arg-
Val19Phe-Asn26His-Gly30Lys
Val4Ile-Val11Ala-Lys17Arg-
Val19Phe-Asn26His-Ile27Phe-
Gly30Lys
Val4Ile-Val11Ala-Lys17Arg-
Val19Phe-Ile27Phe
Val4Ile-Val11Ala-Lys17Arg-
Val19Phe-Gly30Lys
Val4Ile-Val11Ala-Lys17Arg-
Val19Phe-Ile27Phe-Gly30Lys
Val4Ile-Val11Ala-Lys17Arg-
Asn26His-Ile27Phe
Val4Ile-Val11Ala-Lys17Arg-
Asn26His-Gly30Lys
Val4Ile-Val11Ala-Lys17Arg-
Asn26His-Ile27Phe-Gly30Lys
Val4Ile-Val11Ala-Lys17Arg-Ile27Phe
Val4Ile-Val11Ala-Lys17Arg-Gly30Lys
Val4Ile-Val11Ala-Lys17Arg-
Ile27Phe-Gly30Lys
Val4Ile-Val11Ala-Val19Phe-
Asn26His-Ile27Phe
Val4Ile-Val11Ala-Val19Phe-
Asn26His-Gly30Lys
Val4Ile-Val11Ala-Val19Phe-
Asn26His-Ile27Phe-Gly30Lys
Val4Ile-Val11Ala-Val19Phe-Ile27Phe
Leu5Val-Dha8Dhb-Val11Ala-Tyr15Leu-
Lys17Arg-Val19Phe-Asn26His-
Gly30Lys
Leu5Val-Dha8Dhb-Val11Ala-Tyr15Leu-
Lys17Arg-Val19Phe-Asn26His-
Ile27Phe-Gly30Lys
Leu5Val-Dha8Dhb-Val11Ala-Tyr15Leu-
Lys17Arg-Val19Phe-Ile27Phe
Leu5Val-Dha8Dhb-Val11Ala-Tyr15Leu-
Lys17Arg-Val19Phe-Gly30Lys
Leu5Val-Dha8Dhb-Val11Ala-Tyr15Leu-
Lys17Arg-Val19Phe-Ile27Phe-
Gly30Lys
Leu5Val-Dha8Dhb-Val11Ala-Tyr15Leu-
Lys17Arg-Asn26His-Ile27Phe
Leu5Val-Dha8Dhb-Val11Ala-Tyr15Leu-
Lys17Arg-Asn26His-Gly30Lys
Leu5Val-Dha8Dhb-Val11Ala-Tyr15Leu-
Lys17Arg-Asn26His-Ile27Phe-
Gly30Lys
Leu5Val-Dha8Dhb-Val11Ala-Tyr15Leu-
Lys17Arg-Ile27Phe
Leu5Val-Dha8Dhb-Val11Ala-Tyr15Leu-
Lys17Arg-Gly30Lys
Leu5Val-Dha8Dhb-Val11Ala-Tyr15Leu-
Lys17Arg-Ile27Phe-Gly30Lys
Leu5Val-Dha8Dhb-Val11Ala-Tyr15Leu-
Val19Phe-Asn26His-Ile27Phe
Leu5Val-Dha8Dhb-Val11Ala-Tyr15Leu-
Val19Phe-Asn26His-Gly30Lys
Leu5Val-Dha8Dhb-Val11Ala-Tyr15Leu-
Ile27Phe
Leu5Val-Dha8Dhb-Tyr15Leu-Lys17Arg-
Gly30Lys
Leu5Val-Dha8Dhb-Tyr15Leu-Lys17Arg-
Ile27Phe-Gly30Lys
Leu5Val-Dha8Dhb-Tyr15Leu-Val19Phe-
Asn26His-Ile27Phe
Leu5Val-Dha8Dhb-Tyr15Leu-Val19Phe-
Asn26His-Gly30Lys
Leu5Val-Dha8Dhb-Tyr15Leu-Val19Phe-
Asn26His-Ile27Phe-Gly30Lys
Leu5Val-Dha8Dhb-Tyr15Leu-Val19Phe-
Ile27Phe
Leu5Val-Dha8Dhb-Tyr15Leu-Val19Phe-
Gly30Lys
Leu5Val-Dha8Dhb-Tyr15Leu-Val19Phe-
Ile27Phe-Gly30Lys
Leu5Val-Dha8Dhb-Tyr15Leu-Asn26His-
Ile27Phe
Leu5Val-Dha8Dhb-Tyr15Leu-Asn26His-
Gly30Lys
Leu5Val-Dha8Dhb-Tyr15Leu-Asn26His-
Ile27Phe-Gly30Lys
Leu5Val-Dha8Dhb-Tyr15Leu-Ile27Phe
Leu5Val-Dha8Dhb-Tyr15Leu-Gly30Lys
Leu5Val-Dha8Dhb-Tyr15Leu-Ile27Phe-
Gly30Lys
Leu5Val-Dha8Dhb-Lys17Arg-Val19Phe-
Asn26His-Ile27Phe
Leu5Val-Dha8Dhb-Lys17Arg-Val19Phe-
Asn26His-Gly30Lys
Leu5Val-Val11Ala-Tyr15Leu-
Ile27Phe-Gly30Lys
Leu5Val-Val11Ala-Lys17Arg-
Val19Phe-Asn26His-Ile27Phe
Leu5Val-Val11Ala-Lys17Arg-
Val19Phe-Asn26His-Gly30Lys
Leu5Val-Val11Ala-Lys17Arg-
Val19Phe-Asn26His-Ile27Phe-
Gly30Lys
Leu5Val-Val11Ala-Lys17Arg-
Val19Phe-Ile27Phe
Leu5Val-Val11Ala-Lys17Arg-
Val19Phe-Gly30Lys
Leu5Val-Val11Ala-Lys17Arg-
Val19Phe-Ile27Phe-Gly30Lys
Leu5Val-Val11Ala-Lys17Arg-
Asn26His-Ile27Phe
Leu5Val-Val11Ala-Lys17Arg-
Asn26His-Gly30Lys
Leu5Val-Val11Ala-Lys17Arg-
Asn26His-Ile27Phe-Gly30Lys
Leu5Val-Val11Ala-Lys17Arg-Ile27Phe
Leu5Val-Val11Ala-Lys17Arg-Gly30Lys
Leu5Val-Val11Ala-Lys17Arg-
Ile27Phe-Gly30Lys
Leu5Val-Val11Ala-Val19Phe-
Asn26His-Ile27Phe
Leu5Val-Val11Ala-Val19Phe-
Asn26His-Gly30Lys
Leu5Val-Val11Ala-Val19Phe-
Asn26His-Ile27Phe-Gly30Lys
Dha8Dhb-Val11Ala-Tyr15Leu-
Lys17Arg-Val19Phe-Asn26His-
Ile27Phe
Dha8Dhb-Val11Ala-Tyr15Leu-
Lys17Arg-Val19Phe-Asn26His-
Gly30Lys
Dha8Dhb-Val11Ala-Tyr15Leu-
Lys17Arg-Val19Phe-Asn26His-
Ile27Phe-Gly30Lys
Dha8Dhb-Val11Ala-Tyr15Leu-
Lys17Arg-Val19Phe-Ile27Phe
Dha8Dhb-Val11Ala-Tyr15Leu-
Lys17Arg-Val19Phe-Gly30Lys
Dha8Dhb-Val11Ala-Tyr15Leu-
Lys17Arg-Val19Phe-Ile27Phe-
Gly30Lys
Dha8Dhb-Val11Ala-Tyr15Leu-
Lys17Arg-Asn26His-Ile27Phe
Dha8Dhb-Val11Ala-Tyr15Leu-
Lys17Arg-Asn26His-Gly30Lys
Dha8Dhb-Val11Ala-Tyr15Leu-
Lys17Arg-Asn26His-Ile27Phe-
Gly30Lys
Dha8Dhb-Val11Ala-Tyr15Leu-
Lys17Arg-Ile27Phe
Dha8Dhb-Val11Ala-Tyr15Leu-
Lys17Arg-Gly30Lys
Dha8Dhb-Val11Ala-Tyr15Leu-
Lys17Arg-Ile27Phe-Gly30Lys
Dha8Dhb-Val11Ala-Tyr15Leu-
Val19Phe-Asn26His-Ile27Phe
Dha8Dhb-Tyr15Leu-Val19Phe-
Asn26His-Ile27Phe-Gly30Lys
Dha8Dhb-Tyr15Leu-Val19Phe-Ile27Phe
Dha8Dhb-Tyr15Leu-Val19Phe-Gly30Lys
Dha8Dhb-Tyr15Leu-Val19Phe-
Ile27Phe-Gly30Lys
Dha8Dhb-Tyr15Leu-Asn26His-Ile27Phe
Dha8Dhb-Tyr15Leu-Asn26His-Gly30Lys
Dha8Dhb-Tyr15Leu-Asn26His-
Ile27Phe-Gly30Lys
Dha8Dhb-Tyr15Leu-Ile27Phe
Dha8Dhb-Tyr15Leu-Gly30Lys
Dha8Dhb-Tyr15Leu-Ile27Phe-Gly30Lys
Dha8Dhb-Lys17Arg-Val19Phe-
Asn26His-Ile27Phe
Dha8Dhb-Lys17Arg-Val19Phe-
Asn26His-Gly30Lys
Dha8Dhb-Lys17Arg-Val19Phe-
Asn26His-Ile27Phe-Gly30Lys
Dha8Dhb-Lys17Arg-Val19Phe-Ile27Phe
Dha8Dhb-Lys17Arg-Val19Phe-Gly30Lys
Dha8Dhb-Lys17Arg-Val19Phe-
Ile27Phe-Gly30Lys
Dha8Dhb-Lys17Arg-Asn26His-Ile27Phe
Dha8Dhb-Lys17Arg-Asn26His-Gly30Lys
Dha8Dhb-Lys17Arg-Asn26His-
Ile27Phe-Gly30Lys
Dha8Dhb-Lys17Arg-Ile27Phe
Dha8Dhb-Lys17Arg-Gly30Lys
Dha8Dhb-Lys17Arg-Ile27Phe-Gly30Lys
Dha8Dhb-Val19Phe-Asn26His-Ile27Phe
Dha8Dhb-Val19Phe-Asn26His-Gly30Lys
Ile27Phe-Gly30Lys
Tyr15Leu-Lys17Arg-Val19Phe-
Ile27Phe
Tyr15Leu-Lys17Arg-Val19Phe-
Gly30Lys
Tyr15Leu-Lys17Arg-Val19Phe-
Ile27Phe-Gly30Lys
Tyr15Leu-Lys17Arg-Asn26His-
Ile27Phe
Tyr15Leu-Lys17Arg-Asn26His-
Gly30Lys
Tyr15Leu-Lys17Arg-Asn26His-
Ile27Phe-Gly30Lys
Tyr15Leu-Lys17Arg-Ile27Phe
Tyr15Leu-Lys17Arg-Gly30Lys
Tyr15Leu-Lys17Arg-Ile27Phe-
Gly30Lys
Tyr15Leu-Val19Phe-Asn26His-
Ile27Phe
Tyr15Leu-Val19Phe-Asn26His-
Gly30Lys
Tyr15Leu-Val19Phe-Asn26His-
Ile27Phe-Gly30Lys
Tyr15Leu-Val19Phe-Ile27Phe
Tyr15Leu-Val19Phe-Gly30Lys
Tyr15Leu-Val19Phe-Ile27Phe-
Gly30Lys
Tyr15Leu-Asn26His-Ile27Phe
Tyr15Leu-Asn26His-Gly30Lys
Tyr15Leu-Asn26His-Ile27Phe-
Gly30Lys
Val4Ile-Leu5Val-Dha8Dhb-
Val11Ala-Val19Phe-Asn26His-
Ile27Phe
Val4Ile-Leu5Val-Dha8Dhb-
Val11Ala-Val19Phe-Asn26His-
Gly30Lys
Val4Ile-Leu5Val-Dha8Dhb-
Val11Ala-Val19Phe-Asn26His-
Ile27Phe-Gly30Lys
Val4Ile-Leu5Val-Dha8Dhb-
Val11Ala-Val19Phe-Ile27Phe
Val4Ile-Leu5VaL-Dha8Dhb-
Val11Ala-Val19Phe-Gly30Lys
Val4Ile-Leu5Val-Dha8Dhb-
Val11Ala-Val19Phe-Ile27Phe-
Gly30Lys
Val4Ile-Leu5Val-Dha8Dhb-
Val11Ala-Asn26His-Ile27Phe
Val4Ile-Leu5Val-Dha8Dhb-
Val11Ala-Asn26His-Gly30Lys
Val4Ile-Leu5Val-Dha8Dhb-
Val11Ala-Asn26His-Ile27Phe-
Gly30Lys
Val4Ile-Leu5Val-Dha8Dhb-
Val11Ala-Ile27Phe
Val4Ile-Leu5Val-Dha8Dhb-
Val11Ala-Gly30Lys
Val4Ile-Leu5Val-Dha8Dhb-
Val4Ile-Leu5Val-Dha8Dhb-
Val19Phe-Ile27Phe-Gly30Lys
Val4Ile-Leu5Val-Dha8Dhb-
Asn26His-Ile27Phe
Val4Ile-Leu5Val-Dha8Dhb-
Asn26His-Gly30Lys
Val4Ile-Leu5Val-Dha8Dhb-
Asn26His-Ile27Phe-Gly30Lys
Val4Ile-Leu5Val-Dha8Dhb-
Ile27Phe
Val4Ile-Leu5Val-Dha8Dhb-
Gly30Lys
Val4Ile-Leu5Val-Dha8Dhb-
Ile27Phe-Gly30Lys
Val4Ile-Leu5Val-Val11Ala-
Tyr15Leu-Lys17Arg-Val19Phe-
Asn26His-Ile27Phe
Val4Ile-Leu5Val-Val11Ala-
Tyr15Leu-Lys17Arg-Val19Phe-
Asn26His-Gly30Lys
Val4Ile-Leu5Val-Val11Ala-
Tyr15Leu-Lys17Arg-Val19Phe-
Asn26His-Ile27Phe-Gly30Lys
Val4Ile-Leu5Val-Val11Ala-
Tyr15Leu-Lys17Arg-Val19Phe-
Ile27Phe
Val4Ile-Leu5Val-Val11Ala-
Tyr15Leu-Lys17Arg-Val19Phe-
Gly30Lys
Val4Ile-Leu5Val-Val11Ala-
Val4Ile-Leu5Val-Tyr15Leu-
Lys17Arg-Val19Phe-Asn26His-
Gly30Lys
Val4Ile-Leu5Val-Tyr15Leu-
Lys17Arg-Val19Phe-Asn26His-
Ile27Phe-Gly30Lys
Val4Ile-Leu5Val-Tyr15Leu-
Lys17Arg-Val19Phe-Ile27Phe
Val4Ile-Leu5Val-Tyr15Leu-
Lys17Arg-Val19Phe-Gly30Lys
Val4Ile-Leu5Val-Tyr15Leu-
Lys17Arg-Val19Phe-Ile27Phe-
Gly30Lys
Val4Ile-Leu5Val-Tyr15Leu-
Lys17Arg-Asn26His-Ile27Phe
Val4Ile-Leu5Val-Tyr15Leu-
Lys17Arg-Asn26His-Gly30Lys
Val4Ile-Leu5Val-Tyr15Leu-
Lys17Arg-Asn26His-Ile27Phe-
Gly30Lys
Val4Ile-Leu5Val-Tyr15Leu-
Lys17Arg-Ile27Phe
Val4Ile-Leu5Val-Tyr15Leu-
Lys17Arg-Gly30Lys
Val4Ile-Leu5Val-Tyr15Leu-
Lys17Arg-Ile27Phe-Gly30Lys
Val4Ile-Leu5Val-Tyr15Leu-
Val19Phe-Asn26His-Ile27Phe
Val4Ile-Leu5Val-Tyr15Leu-
Val19Phe-Asn26His-Gly30Lys
Val4Ile-Leu5Val-Tyr15Leu-
Val19Phe-Asn26His-Ile27Phe-
Gly30Lys
Val4Ile-Dha8Dhb-Val11Ala-
Tyr15Leu-Val19Phe-Ile27Phe
Val4Ile-Dha8Dhb-Val11Ala-
Tyr15Leu-Val19Phe-Gly30Lys
Val4Ile-Dha8Dhb-Val11Ala-
Tyr15Leu-Val19Phe-Ile27Phe-
Gly30Lys
Val4Ile-Dha8Dhb-Val11Ala-
Tyr15Leu-Asn26His-Ile27Phe
Val4Ile-Dha8Dhb-Val11Ala-
Tyr15Leu-Asn26His-Gly30Lys
Val4Ile-Dha8Dhb-Val11Ala-
Tyr15Leu-Asn26His-Ile27Phe-
Gly30Lys
Val4Ile-Dha8Dhb-Val11Ala-
Tyr15Leu-Ile27Phe
Val4Ile-Dha8Dhb-Val11Ala-
Tyr15Leu-Gly30Lys
Val4Ile-Dha8Dhb-Val11Ala-
Tyr15Leu-Ile27Phe-Gly30Lys
Val4Ile-Dha8Dhb-Val11Ala-
Lys17ArgVal19Phe-Asn26His-
Ile27Phe
Val4Ile-Dha8Dhb-Val11Ala-
Lys17Arg-Val19Phe-Asn26His-
Gly30Lys
Val4Ile-Dha8Dhb-Val11Ala-
Lys17Arg-Val19Phe-Asn26His-
Ile27Phe-Gly30Lys
Asn26His-Gly30Lys
Val4Ile-Dha8Dhb-Lys17Arg-
Val19Phe-Asn26His-Ile27Phe-
Gly30Lys
Val4Ile-Dha8Dhb-Lys17Arg-
Val19Phe-Ile27Phe
Val4Ile-Dha8Dhb-Lys17Arg-
Val19Phe-Gly30Lys
Val4Ile-Dha8Dhb-Lys17Arg-
Val19Phe-Ile27Phe-Gly30Lys
Val4Ile-Dha8Dhb-Lys17Arg-
Asn26His-Ile27Phe
Val4Ile-Dha8Dhb-Lys17Arg-
Asn26His-Gly30Lys
Val4Ile-Dha8Dhb-Lys17Arg-
Asn26His-Ile27Phe-Gly30Lys
Val4Ile-Dha8Dhb-Lys17Arg-
Ile27Phe
Val4Ile-Dha8Dhb-Lys17Arg-
Gly30Lys
Val4Ile-Dha8Dhb-Lys17Arg-
Ile27Phe-Gly30Lys
Val4Ile-Dha8Dhb-Val19Phe-
Asn26His-Ile27Phe
Val4Ile-Dha8Dhb-Val19Phe-
Asn26His-Gly30Lys
Val4Ile-Dha8Dhb-Val19Phe-
Asn26His-Ile27Phe-Gly30Lys
Val4Ile-Dha8Dhb-Val19Phe-
Ile27Phe
Val4Ile-Dha8Dhb-Val19Phe-
Val4Ile-Val11Ala-Val19Phe-
Gly30Lys
Val4Ile-Val11Ala-Val19Phe-
Ile27Phe-Gly30Lys
Val4Ile-Val11Ala-Asn26His-
Ile27Phe
Val4Ile-Val11Ala-Asn26His-
Gly30Lys
Val4Ile-Val11Ala-Asn26His-
Ile27Phe-Gly30Lys
Val4Ile-Val11Ala-Ile27Phe
Val4Ile-Val11Ala-Gly30Lys
Val4Ile-Val11Ala-Ile27Phe-
Gly30Lys
Val4Ile-Tyr15Leu-Lys17Arg-
Val19Phe-Asn26His-Ile27Phe
Val4Ile-Tyr15Leu-Lys17Arg-
Val19Phe-Asn26His-Gly30Lys
Val4Ile-Tyr15Leu-Lys17Arg-
Val19Phe-Asn26His-Ile27Phe-
Gly30Lys
Val4Ile-Tyr15Leu-Lys17Arg-
Val19Phe-Ile27Phe
Val4Ile-Tyr15Leu-Lys17Arg-
Val19Phe-Gly30Lys
Val4Ile-Tyr15Leu-Lys17Arg-
Val19Phe-Ile27Phe-Gly30Lys
Val4Ile-Tyr15Leu-Lys17Arg-
Asn26His-Ile27Phe
Val4Ile-Tyr15Leu-Lys17Arg-
Asn26His-Gly30Lys
Val19Phe-Asn26His-Ile27Phe-
Gly30Lys
Leu5Val-Dha8Dhb-Val11Ala-
Tyr15Leu-Val19Phe-Ile27Phe
Leu5Val-Dha8Dhb-Val11Ala-
Tyr15Leu-Val19Phe-Gly30Lys
Leu5Val-Dha8Dhb-Val11Ala-
Tyr15Leu-Val19Phe-Ile27Phe-
Gly30Lys
Leu5Val-Dha8Dhb-Val11Ala-
Tyr15Leu-Asn26His-Ile27Phe
Leu5Val-Dha8Dhb-Val11Ala-
Tyr15Leu-Asn26His-Gly30Lys
Leu5Val-Dha8Dhb-Val11Ala-
Tyr15Leu-Asn26His-Ile27Phe-
Gly30Lys
Leu5Val-Dha8Dhb-Val11Ala-
Tyr15Leu-Ile27Phe
Leu5Val-Dha8Dhb-Val11Ala-
Tyr15Leu-Gly30Lys
Leu5Val-Dha8Dhb-Val11Ala-
Tyr15Leu-Ile27Phe-Gly30Lys
Leu5Val-Dha8Dhb-Val11Ala-
Lys17Arg-Val19Phe-Asn26His-
Ile27Phe
Leu5Val-Dha8Dhb-Val11Ala-
Lys17Arg-Val19Phe-Asn26His-
Gly30Lys
Leu5Val-Dha8Dhb-Val11Ala-
Lys17Arg-Val19Phe-Asn26His-
Ile27Phe-Gly30Lys
Leu5Val-Dha8Dhb-Lys17Arg-
Val19Phe-Asn26His-Ile27Phe-
Gly30Lys
Leu5Val-Dha8Dhb-Lys17Arg-
Val19Phe-Ile27Phe
Leu5Val-Dha8Dhb-Lys17Arg-
Val19Phe-Gly30Lys
Leu5Val-Dha8Dhb-Lys17Arg-
Val19Phe-Ile27Phe-Gly30Lys
Leu5Val-Dha8Dhb-Lys17Arg-
Asn26His-Ile27Phe
Leu5Val-Dha8Dhb-Lys17Arg-
Asn26His-Gly30Lys
Leu5Val-Dha8Dhb-Lys17Arg-
Asn26His-Ile27Phe-Gly30Lys
Leu5Val-Dha8Dhb-Lys17Arg-
Ile27Phe
Leu5Val-Dha8Dhb-Lys17Arg-
Gly30Lys
Leu5Val-Dha8Dhb-Lys17Arg-
Ile27Phe-Gly30Lys
Leu5Val-Dha8Dhb-Val19Phe-
Asn26His-Ile27Phe
Leu5Val-Dha8Dhb-Val19Phe-
Asn26His-Gly30Lys
Leu5Val-Dha8Dhb-Val19Phe-
Asn26His-Ile27Phe-Gly30Lys
Leu5Val-Dha8Dhb-Val19Phe-
Ile27Phe
Leu5Val-Dha8Dhb-Val19Phe-
Gly30Lys
Leu5Val-Val11Ala-Val19Phe-
Ile27Phe
Leu5Val-Val11Ala-Val19Phe-
Gly30Lys
Leu5Val-Val11Ala-Val19Phe-
Ile27Phe-Gly30Lys
Leu5Val-Val11Ala-Asn26His-
Ile27Phe
Leu5Val-Val11Ala-Asn26His-
Gly30Lys
Leu5Val-Val11Ala-Asn26His-
Ile27Phe-Gly30Lys
Leu5Val-Val11Ala-Ile27Phe
Leu5Val-Val11Ala-Gly30Lys
Leu5Val-Val11Ala-Ile27Phe-
Gly30Lys
Leu5Val-Tyr15Leu-Lys17Arg-
Val19Phe-Asn26His-Ile27Phe
Leu5Val-Tyr15Leu-Lys17Arg-
Val19Phe-Asn26His-Gly30Lys
Leu5Val-Tyr15Leu-Lys17Arg-
Val19Phe-Asn26His-Ile27Phe-
Gly30Lys
Leu5Val-Tyr15Leu-Lys17Arg-
Val19Phe-Ile27Phe
Leu5Val-Tyr15Leu-Lys17Arg-
Val19Phe-Gly30Lys
Leu5Val-Tyr15Leu-Lys17Arg-
Val19Phe-Ile27Phe-Gly30Lys
Leu5Val-Tyr15Leu-Lys17Arg-
Asn26His-Ile27Phe
Dha8Dhb-Val11Ala-Tyr15Leu-
Val19Phe-Asn26His-Gly30Lys
Dha8Dhb-Val11Ala-Tyr15Leu-
Val19Phe-Asn26His-Ile27Phe-
Gly30Lys
Dha8Dhb-Val11Ala-Tyr15Leu-
Val19Phe-Ile27Phe
Dha8Dhb-Val11Ala-Tyr15Leu-
Val19Phe-Gly30Lys
Dha8Dhb-Val11Ala-Tyr15Leu-
Val19Phe-Ile27Phe-Gly30Lys
Dha8Dhb-Val11Ala-Tyr15Leu-
Asn26His-Ile27Phe
Dha8Dhb-Val11Ala-Tyr15Leu-
Asn26His-Gly30Lys
Dha8Dhb-Val11Ala-Tyr15Leu-
Asn26His-Ile27Phe-Gly30Lys
Dha8Dhb-Val11Ala-Tyr15Leu-
Ile27Phe
Dha8Dhb-Val11Ala-Tyr15Leu-
Gly30Lys
Dha8Dhb-Val11Ala-Tyr15Leu-
Ile27Phe-Gly30Lys
Dha8Dhb-Val11Ala-Lys17Arg-
Val19Phe-Asn26His-Ile27Phe
Dha8Dhb-Val11Ala-Lys17Arg-
Val19Phe-Asn26His-Gly30Lys
Dha8Dhb-Val11Ala-Lys17Arg-
Val19Phe-Asn26His-Ile27Phe-
Gly30Lys
Dha8Dhb-Val11Ala-Lys17Arg-
Dha8Dhb-Val19Phe-Asn26His-
Ile27Phe-Gly30Lys
Dha8Dhb-Val19Phe-Ile27Phe
Dha8Dhb-Val19Phe-Gly30Lys
Dha8Dhb-Val19Phe-Ile27Phe-
Gly30Lys
Dha8Dhb-Asn26His-Ile27Phe
Dha8Dhb-Asn26His-Gly30Lys
Dha8Dhb-Asn26His-Ile27Phe-
Gly30Lys
Dha8Dhb-Ile27Phe
Dha8Dhb-Gly30Lys
Dha8Dhb-Ile27Phe-Gly30Lys
Val11Ala-Tyr15Leu-Lys17Arg-
Val19Phe-Asn26His-Ile27Phe
Val11Ala-Tyr15Leu-Lys17Arg-
Val19Phe-Asn26His-Gly30Lys
Val11Ala-Tyr15Leu-Lys17Arg-
Val19Phe-Asn26His-Ile27Phe-
Gly30Lys
Val11Ala-Tyr15Leu-Lys17Arg-
Val19Phe-Ile27Phe
Val11Ala-Tyr15Leu-Lys17Arg-
Val19Phe-Gly30Lys
Val11Ala-Tyr15Leu-Lys17Arg-
Val19Phe-Ile27Phe-Gly30Lys
Val11Ala-Tyr15Leu-Lys17Arg-
Asn26His-Ile27Phe
Val11Ala-Tyr15Leu-Lys17Arg-
Asn26His-Gly30Lys
Val11Ala-Tyr15Leu-Lys17Arg-
Asn26His-Ile27Phe-Gly30Lys
Tyr15Leu-Ile27Phe
Tyr15Leu-Gly30Lys
Tyr15Leu-Ile27Phe-Gly30Lys
Lys17Arg-Val19Phe-Asn26His-
Ile27Phe
Lys17Arg-Val19Phe-Asn26His-
Gly30Lys
Lys17Arg-Val19Phe-Asn26His-
Ile27Phe-Gly30Lys
Lys17Arg-Val19Phe-Ile27Phe
Lys17Arg-Val19Phe-Gly30Lys
Lys17Arg-Val19Phe-Ile27Phe-
Gly30Lys
Lys17Arg-Asn26His-Ile27Phe
Lys17Arg-Asn26His-Gly30Lys
Lys17Arg-Asn26His-Ile27Phe-
Gly30Lys
Lys17Arg-Ile27Phe
Lys17Arg-Gly30Lys
Lys17Arg-Ile27Phe-Gly30Lys
Val19Phe-Asn26His-Ile27Phe
Val19Phe-Asn26His-Gly30Lys
Val19Phe-Asn26His-Ile27Phe-
Gly30Lys
Val19Phe-Ile27Phe
Val19Phe-Gly30Lys
Val19Phe-Ile27Phe-Gly30Lys
Asn26His-Ile27Phe
Asn26His-Gly30Lys
Asn26His-Ile27Phe-Gly30Lys
Ile27Phe-Gly30Lys

1-16. (canceled)

17. An antimicrobial compound termed epilancin 15X having an amino acid sequence set forth in SEQ ID NO: 1 [X-Ala-U-Ile-Val-Lys-O-O-Ile-Lys-Ala-Ala-Lys-Lys-Leu-Ala-Arg-Gly-Phe-A*-Leu-A*-Ala-Gly-Ala-His-Phe-O-Gly-Lys-Lys in which X is hydroxy propionyl (Hop), U is α,β-didehydroalanine (Dha), O is α,β-didehydrobutyrine (Dhb), A* is aminobutyrine (Abu)], wherein lanthionine ring structures are formed between Ala¹² and Ala¹⁶, between Abu²⁰ and Ala²³ and between Abu²² and Ala²⁵.

18. A pharmaceutical composition for the treatment of microbial infections comprising the antimicrobial compound of claim 17 and a suitable excipient.

19. A modified antimicrobial compound comprising from one to nine amino acid substitutions of an amino acid sequence set forth in SEQ ID NO:2 [X-Ala-U-Val-Leu-Lys-O-U-Ile-Lys-Val-Ala-Lys-Lys-Tyr-Ala-Lys-Gly-Val-A*-Leu-A*-Ala-Gly-Ala-Asn-Ile-O-Gly-Gly-Lys in which X is hydroxy propionyl (Hop), U is α,β-didehydroalanine (Dha), O is α,β-didehydrobutyrine (Dhb), A* is aminobutyrine (Abu)], wherein lanthionine ring structures are formed between Ala¹² and Ala¹⁶, between Abu²⁰ and Ala²³ and between Abu²² and Ala²⁵.

20. The antimicrobial compound of claim 19 having antimicrobial activity against Staphylococcus epidermidis ATCC strain 49134.

21. The antimicrobial compound of claim 20, wherein said from one to nine amino acid substitutions are at from one to nine positions selected from the group consisting of 4, 5, 8, 11, 15, 17, 19, 26, 27 and 30.

22. The antimicrobial compound of claim 21, comprising from one to nine amino acid substitutions selected from the group consisting of Val⁴Ile, Leu⁵Val, Dha⁸Dhb,Val¹¹Ala,Tyr¹⁵Leu, Lys¹⁷Arg,Val¹⁹Phe, Asn²⁶His, Ile²⁷Phe and Gly³⁰Ly.

23. The antimicrobial compound of claim 19 having a sequence homology of at least 71% with SEQ ID NO:1.

24. The antimicrobial compound of claim 19 having a sequence homology of at least 74% with SEQ ID NO:1.

25. The antimicrobial compound of claim 19 having a sequence homology of at least 77% with SEQ ID NO:1.

26. The antimicrobial compound of claim 19 having a sequence homology of at least 81% with SEQ ID NO:1.

27. The antimicrobial compound of claim 19 having a sequence homology of at least 84% with SEQ ID NO:1.

28. The antimicrobial compound of claim 19 having a sequence homology of at least 87% with SEQ ID NO:1.

29. The antimicrobial compound of claim 19 having a sequence homology of at least 90% with SEQ ID NO:1.

30. The antimicrobial compound of claim 19 having a sequence homology of at least 94% with SEQ ID NO:1.

31. The antimicrobial compound of claim 19 having a sequence homology of at least 97% with SEQ ID NO:1.

32. The antimicrobial compound of claim 19 having essentially the same antimicrobial activity as epilancin X15.

33. A method for obtaining an antimicrobial compound comprising:
a) growing Staphylococcus epidermidis strain 15x154 (CBS accession no. 113428) in growth medium;
b) removing cells of said Staphylococcus epidermidis strain 15x154 (CBS accession no. 113428) to obtain a supernatant;
c) passing the supernatant over a cation exchange liquid chromatography column;
d) eluting fractions and determining their antimicrobial activity;
e) pooling the fractions showing antimicrobial activity and passing them over a hydrophobic interaction column;
f) eluting fractions and determining their antimicrobial activity;
g) pooling the fractions showing antimicrobial activity and passing them over a reverse phase liquid chromatography column;
h) eluting fractions and determining their antimicrobial activity; and
i) pooling the active fractions and concentrating an antimicrobial compound having a molecular weight of about 3100 Daltons contained therein.

34. The method of claim 33, wherein the growth medium is Mueller Hinton medium.

35. The antimicrobial compound produced using the method of claim 34, wherein said antimicrobial activity is tested against Staphylococcus epidermidis ATCC strain 49134.

36. A method of treating a microbial infection, comprising administering the antimicrobial compound of claim 35, to a subject having a microbial infection.
